(19) United States
(12) Patent Application Publication
(10) Pub. No.: US 2002/0039734 A1
(43) Pub. Date: Apr. 4, 2002

(54) Compositions, kits, and methods for identification and modulation of T helper-1 and T helper-2 cells and diseases associated therewith

(76) Inventors: Catherine F. Hanrahan, London (GB); Marc Feldmann, London (GB); William L. Trepicchio, Andover, MA (US)

Correspondence Address: LAHIVE & COCKFIELD, 28 STATE STREET, BOSTON, MA, 02109, US

(21) Appl. No.: 09/860,655

(22) Filed: May 17, 2001

Related U.S. Application Data
(60) Provisional application No. 60/205,204, filed on May 18, 2000.

(51) Int. Cl. C12Q 1/68; G01N 33/574

(52) U.S. Cl. 435/6; 435/7.23

(57) ABSTRACT

The invention relates to compositions, kits and methods for identifying, detecting, and modulating the differentiation, growth, and/or maturation of Th1 or Th2 cells. The invention further relates to compositions, kits, and methods for detecting, characterizing, preventing, and treating a Th1- or Th2-associated condition. A variety of markers are provided, wherein changes in the levels of expression of one or more of the markers is correlated with the presence of a Th1 or Th2 cell or Th1- or Th2-associated condition.

### RELATED APPLICATIONS

This application claims priority to U.S. Provisional Application No.: 60/205,204 filed on May 18, 2000, incorporated herein in it's entirety by this reference.

### BACKGROUND OF THE INVENTION

The development of effector CD4⁺ T cells from naïve T cells is an important step in the generation of an effective and appropriate immune response to an antigen. The activation of precursor CD4⁺ T cells occurs when the T cell receptor recognizes a specific antigen that is presented in the context of MHC class II molecules on antigen presenting cells (APC). It has long been recognized that heterogeneous immune responses are generated in response to different pathogens and pathological conditions. The discovery of two different types of CD4⁺ effector T cells defined by their cytokine production profile partially explained the observed heterogeneity in immune responses (Mosmann et al. (1986) J Immunol 136(7):2348-57). T helper I cells (Th1 cells) specifically produce IFN-γ and IL-2, the first of which is the dominant cytokine involved in generating a cell-mediated immune response that is generally elicited in response to intracellular pathogens. In addition, Th1 cells contribute to the pathology of inflammatory diseases such as inflammatory bowel disease, rheumatoid arthritis, multiple sclerosis, and psoriasis (Liblau et al. (1995) Immunol Today 1995 16(1):34-8). Th2 cells specifically produce IL-4, IL-5 and IL-13. These cytokines are able to induce B cell growth and differentiation and provide T cell help for a humoral immune response. Th2 effector cells have been associated with disease states such as allergy and asthma (Liblau et al. (1995) Immunol Today 1995 16(l):34-8).

Since the identification of two T helper cell subsets more than 10 years ago, much attention has focused on the molecules that induce differentiation of these two cell types from a common naïve precursor. In vitro analysis of Th cell generation from a naïve CD4⁺ T cell precursor has been instrumental in defining some of the molecules that are crucial for differentiation. IL-12, produced primary by macrophages and dendritic cells in vivo, has been shown to induce differentiation of naïve CD4⁺ T cells into Th1 cells, which are characterized by the production of large amounts of IFN-γ (Hsieh (1993) Science 260: 547-549; Seder et al. (1993) Proc Natl Acad Sci USA 90(21):10188-92; Manetti et al. (1993) J Exp Med 177(4):1199-204). Th2 differentiation is induced by IL-4 but the initial source of IL-4 that triggers Th2 differentiation is still uncertain. Potential cellular sources of IL-4 include basophils, mast cells and NK1.1 CD4⁺ T cells, all of which are present in Th2-mediated cellular immune responses (Seder (1991) Proc. Natl. Acad. Sci. USA 88: 2835-2839; Plaut (1989) Nature 339: 64-7; Scott (1990) J. Immunol. 145: 2183-2188).

In recent years, details of the signaling pathways leading to Th differentiation have begun to emerge. Signal transducer and activator of transcription (STAT)-3 and STAT4 are transcription factors activated by IL-12 and involved in Th1 differentiation, whereas Th2 differentiation has been shown to depend on IL-4 activation of STAT6 (Jacobson (1995) J. Exp. Med. 181:1755-62; Kaplan (1996) Immunity 4: 313-9; Thierfelder (1996) Nature 382(6587):171-4; Shimoda et al. (1996) Nature 380(6575):630-3; Takeda (1996) Nature 380(6575):627-30). Production of IFN-γ in Th1 cells was shown to be dependent on the mitogen-activated protein kinase JNK2 and p38 (Rincon (1998) EMBO J. 17: 2817-2829). In addition to the STAT molecules, several other transcription factors that are involved in Th differentiation have been identified. The transcription factor ERM was induced by IL-12 in a STAT-4-dependent manner in Th1 cells but could not restore IFN-γ production in STAT-4-deficient T cells (Ouyang (1999) Proc Natl Acad Sci USA 96(7):3888-93). T-bet, a T box transcription factor, was recently shown to control expression of IFN-γ and could redirect Th2 clones to express IFN-γ (Szabo et al (2000) Cell 100(6):655-69). GATA-3, c-maf, NF-IL-6 and NIP-45 transcription factors have all been shown to induce transcription of IL-4 (Zheng (1997) Cell 89: 587-596; Zhang (1997) J Biol. Chem. 272(14):9474-80; Ho (1996) Cell 85: 973-983; Hodge (1996) Science 274: 1903-1905; Davydov (1995) J Immunol 155(11):5273-9). While these studies have provided some insight into the signaling mechanisms that are important in differentiating Th1 and Th2 cells, there remain many unanswered questions. Previous studies have demonstrated that naïve CD4⁺ T cells begin to produce IFN-γ mRNA in response to IL-12 after 6 hours, and IL-4 mRNA in response to exogenous IL-4 after 48 hours (Lederer (1996) J. Exp. Med. 184: 397-406). Therefore it is likely that molecules critical for Th1 and Th2 development will be expressed in the first 24-48 hours of differentiation.

### SUMMARY OF THE INVENTION

In one embodiment, the invention provides a method of assessing whether Th1 or Th2 cells are present in a subject, by comparing the level of expression of a marker in a sample from a subject, where the marker is selected from the group of markers set forth in Tables 2-5 and 8-10, to the normal level of expression of the marker in a control sample, where a significant difference between the level of expression of the marker in the sample from the subject and the normal level is an indication that Th1 or Th2 cells are present in the subject. In a preferred embodiment, the marker corresponds to a transcribed polynucleotide or portion thereof, where the polynucleotide includes the marker. In a particularly preferred embodiment, the level of expression of the marker in the sample differs from the normal level of expression of the marker in naïve T cells by a factor of at least two, and in an even more preferred embodiment, the expression levels differ by a factor of at least five. In another preferred embodiment, the marker is not significantly expressed in tissue lacking Th1 or Th2 cells.

In another preferred embodiment, the sample includes cells obtained from the subject. In another preferred embodiment, the level of expression of the marker in the sample is assessed by detecting the presence in the sample of a protein corresponding to the marker. In a particularly preferred embodiment, the presence of the protein is detected using a reagent which specifically binds with the protein. In an even more preferred embodiment, the reagent is selected from the group of reagents including an antibody, an antibody derivative, and an antibody fragment. In another preferred embodiment, the level of expression of the marker in the sample is assessed by detecting the presence in the sample of a transcribed polynucleotide or portion thereof, where the transcribed polynucleotide includes the marker. In a particularly preferred embodiment, the transcribed polynucleotide is an mRNA or a cDNA. In another particularly preferred embodiment, the step of detecting further comprises amplifying the transcribed polynucleotide.

In yet another preferred embodiment, the level of expression of the marker in the sample is assessed by detecting the presence in the sample of a transcribed polynucleotide which anneals with the marker or anneals with a portion of a polynucleotide under stringent hybridization conditions, where the polynucleotide includes the marker. In another preferred embodiment, the level of expression in the sample of each of a plurality of markers independently selected from the markers listed in Tables 2-5 and 8-10 is compared with the normal level of expression of each of the plurality of markers in samples of the same type obtained from control samples, where the level of expression of more than one of the markers is significantly altered, relative to the corresponding normal levels of expression of the markers, is an indication that Th1 or Th2 cells are present in the sample. In a particularly preferred embodiment, the plurality includes two or more of the markers. In a still more preferred embodiment, the plurality includes at least five of the markers set forth in Tables 2-5 and 8-10.

In another embodiment, the invention provides a method for monitoring the differentiation of naïve T cells into Th1 or Th2 cells in a subject, including detecting in a subject sample at a first point in time the expression of marker, where the marker is selected from the group including the markers listed in Tables 2-5 and 8-10, repeating this detection step at a subsequent point in time, and comparing the level of expression detected in the two detection steps, and monitoring the differentiation of naïve T cells into Th1 or Th2 cells in the subject using this information. In a preferred embodiment, the marker is selected from the group including the markers listed in Tables 2-5 and 8-10 and combinations thereof. In another preferred embodiment, the marker corresponds to a transcribed polynucleotide or portion thereof, where the polynucleotide includes the marker. In another preferred embodiment, the sample includes cells obtained from the subject. In a particularly preferred embodiment, the cells are collected from lymph or blood tissue.

In another embodiment, the invention provides a method of assessing the efficacy of a test compound or therapy for modulating differentiation of Th1 or Th2 cells in a subject, including comparing expression of a marker in a first sample obtained from the subject which is exposed to or maintained in the presence of the test compound or therapy, where the marker is selected from the group including the markers listed in Tables 2-5 and 8-10, to expression of the marker in a second sample obtained from the subject, where the second sample is not exposed to the test compound or therapy, where a significantly lower level of expression of the marker in the first sample relative to that in the second sample is an indication that the test compound or therapy is efficacious for modulating differentiation of Th1 or Th2 cells in the subject. In a preferred embodiment, the first and second samples are portions of a single sample obtained from the subject. In another preferred embodiment, the first and second samples are portions of pooled samples obtained from the subject.

In another embodiment, the invention provides a method of assessing the efficacy of a test compound or therapy for modulating differentiation of Th1 or Th2 cells in a subject, the method including comparing expression of a marker in the first sample obtained from the subject prior to providing at least a portion of the test compound or therapy to the subject, where the marker is selected from the group including the markers listed in Tables 2-5 and 8-10, to expression of the marker in a second sample obtained from the subject following provision of the portion of the test compound or therapy, where a significantly lower level of expression of the marker in the second sample relative to the first sample is an indication that the test compound or therapy is efficacious for modulating differentiation of Th1 or Th2 cells in the subject.

In another embodiment, the invention provides a method of assessing the efficacy of a test compound or therapy for modulating growth or maturation of Th1 or Th2 cells in a subject, the method including comparing expression of a marker in the first sample obtained from the subject prior to providing at least a portion of the test compound or therapy to the subject, where the marker is selected from the group including the markers listed in Tables 2-5 and 8-10, and not including IFNG, SCYA20, or APT1, to expression of the marker in a second sample obtained from the subject following provision of the portion of the test compound or therapy, where a significantly enhanced level of expression of the marker in the first sample relative to the second sample is an indication that the test compound or therapy is efficacious for modulating growth or maturation of Th1 or Th2 cells in a subject.

In another embodiment, the invention provides a method of assessing the efficacy of a test compound or therapy for modulating growth or maturation of Th1 or Th2 cells in a subject, the method including comparing expression of a marker in the first sample obtained from the subject prior to providing at least a portion of the test compound or therapy to the subject, where the marker is selected from the group including the markers listed in Tables 2-5 and 8-10, and not including IFNG, SCYA20, or APT 1, to expression of the marker in a second sample obtained from the subject following provision of the portion of the test compound or therapy, where a significantly enhanced level of expression of the marker in the second sample relative to the first sample is an indication that the test compound or therapy is efficacious for modulating growth or maturation of Th1 or Th2 cells in a subject.

In another embodiment, the invention provides a method of selecting a composition for modulating differentiation of Th1 or Th2 cells in a subject, the method including obtaining a sample including cells from a subject, separately maintaining aliquots of the sample in the presence of a plurality of test compositions, comparing expression of a marker in each of the aliquots, where the marker is selected from the group including the markers listed in Tables 2-5 and 8-10, and selecting one of the test compositions which induces a decreased level of expression of the marker in the aliquot containing that test composition, relative to other test compositions.

In another embodiment, the invention provides a method of selecting a composition for modulating differentiation of Th1 or Th2 cells in a subject, the method including obtaining a sample including cells from a subject, separately maintaining aliquots of the sample in the presence of a plurality of test compositions, comparing expression of a marker in each of the aliquots, where the marker is selected from the group including the markers listed in Tables 2-5 and 8-10, and selecting one of the test compositions which induces an increased level of expression of the marker in the aliquot containing that test composition, relative to other test compositions.

In another embodiment, the invention provides a method of selecting a composition for modulating growth or maturation of Th1 or Th2 cells in a subject, the method including obtaining a sample including cells from a subject, separately maintaining aliquots of the sample in the presence of a plurality of test compositions, comparing expression of a marker in each of the aliquots, where the marker is selected from the group including the markers listed in Tables 2-5 and 8-10, and not including IFNG, SCYA20, or APT1, and selecting one of the test compositions which induces a decreased level of expression of the marker in the aliquot containing that test composition, relative to other test compositions.

In another embodiment, the invention provides a method of selecting a composition for modulating growth or maturation of Th1 or Th2 cells in a subject, the method including obtaining a sample including cells from a subject, separately maintaining aliquots of the sample in the presence of a plurality of test compositions, comparing expression of a marker in each of the aliquots, where the marker is selected from the group including the markers listed in Tables 2-5 and 8-10, and not including IFNG, SCYA20, or APT1, and selecting one of the test compositions which induces an increased level of expression of the marker in the aliquot containing that test composition, relative to other test compositions.

In another embodiment, the invention provides a method of modulating differentiation, growth, or development of Th1 or Th2 cells in a subject, the method including obtaining a sample including cells from a subject, separately maintaining aliquots of the sample in the presence of a plurality of test compositions, comparing expression of a marker in each of the aliquots, where the marker is selected from the group including the markers listed in Tables 2-5 and 8-10, and selecting one of the test compositions which induces an enhanced level of expression of the marker in the aliquot containing that test composition, relative to other test compositions.

In another embodiment, the invention provides a method of modulating differentiation, growth, or development of Th1 or Th2 cells in a subject, the method including obtaining a sample including cells from a subject, separately maintaining aliquots of the sample in the presence of a plurality of test compositions, comparing expression of a marker in each of the aliquots, where the marker is selected from the group including the markers listed in Tables 2-5 and 8-10, and selecting one of the test compositions which induces a decreased level of expression of the marker in the aliquot containing that test composition, relative to other test compositions.

In another embodiment, the invention provides a kit for assessing whether Th1 or Th2 cells are present in a subject, including reagents for assessing expression of a marker selected from the group including the markers listed in Tables 2-5 and 8-10.

In another embodiment, the invention provides a kit for assessing the presence of mature Th1 or Th2 cells, the kit including a nucleic acid probe where the probe specifically binds with a transcribed polynucleotide corresponding to a marker selected from the group including the markers listed in Tables 2-5 and 8-10.

In another embodiment, the invention provides a kit for assessing the presence of Th1 or Th2 cells differentiated for 24 or fewer hours, the kit including a nucleic acid probe where the probe specifically binds with a transcribed polynucleotide corresponding to a marker selected from the group including the markers listed in Tables 2-5 and 8-10, but not including IFNG, SCYA20, or APT1.

In another embodiment, the invention provides a kit for assessing the suitability of each of a plurality of compounds for modulating differentiation of Th1 or Th2 cells in a subject, the kit including a plurality of compounds and a reagent for assessing expression of a marker selected from the group including the markers listed in Tables 2-5 and 8-10.

In another embodiment, the invention provides a kit for assessing the presence of Th1 or Th2 cells in a sample, including an antibody, where the antibody specifically binds with a protein corresponding to a marker selected from the group including the markers listed in Tables 2-5 and 8-10.

In another embodiment, the invention provides a kit for assessing the presence of Th1 or Th2 cells in a sample, the kit including a nucleic acid probe where the prove specifically binds with a transcribed polynucleotide corresponding to a marker selected from the group including the markers listed in Tables 2-5 and 8-10.

In another embodiment, the invention provides a method of assessing the potential of a test compound to trigger the differentiation of Th1 or Th2 cells from naïve T cells, including maintaining separate aliquots of cells in the presence and absence of the test compound, and comparing expression of a marker in each of the aliquots, where the marker is selected from the group including the markers listed in Tables 2-5 and 8-10, where a significantly enhanced level of expression of the marker in the aliquot maintained in the presence of the test compound, relative to the aliquot maintained in the absence of the test compound, is an indication that the test compound possesses the potential for triggering naïve T cells to differentiate into Th1 or Th2 cells.

In another embodiment, the invention provides a method of assessing the potential of a test compound to trigger the differentiation of Th1 or Th2 cells from naïve T cells, including maintaining separate aliquots of cells in the presence and absence of the test compound, and comparing expression of a marker in each of the aliquots, where the marker is selected from the group including the markers listed in Tables 2-5 and 8-10, where a significantly decreased level of expression of the marker in the aliquot maintained in the presence of the test compound, relative to the aliquot maintained in the absence of the test compound, is an indication that the test compound possesses the potential for triggering naïve T cells to differentiate into Th1 or Th2 cells.

In another embodiment, the invention provides a kit for assessing the potential for triggering the differentiation of naïve T cells into Th1 or Th2 cells, including cells and a reagent for assessing expression of a marker, where the marker is selected from the group including the markers listed in Tables 2-5 and 8-10.

In another embodiment, the invention provides a method of treating a subject in which differentiation of naïve T cells into Th₁ and Th2 cells is desired, including providing to cells of the subject afflicted with a Th1- or Th2-associated condition a protein corresponding to a marker selected from the markers listed in Tables 2-5 and 8-10. In a preferred embodiment, the protein is provided to the cells by providing a vector including a polynucleotide encoding the protein to the cells.

In another embodiment, the invention provides a method of treating a subject in which differentiation of naïve T cells into Th1 and Th2 cells is desired an antisense oligonucleotide complementary to a polynucleotide corresponding to a marker selected from the markers listed in Tables 2-5 and 8-10.

In another embodiment, the invention provides a method of inhibiting Th1 or Th2 differentiation in a subject, including inhibiting expression of a gene corresponding to a marker selected from the markers listed in Tables 2-5 and 8-10.

In another embodiment, the invention provides a method of inhibiting Th1 or Th2 differentiation in a subject, the method comprising enhancing expression of a gene corresponding to a marker selected from the markers listed in Tables 2-5 and 8-10.

Other features and advantages of the invention will be apparent from the following detailed description and claims.

### BRIEF DESCRIPTION OF THE DRAWINGS

FIGS. 1A and 1B depict the results of a Taqman 5′ nuclease fluorigenic quantitative PCR assay of MIF mRNA expression in naïve CD4⁺ T cells, in cells exposed to Th1-inducing conditions, and in cells exposed to Th2-inducing conditions. MIF mRNA expression was measured in three individual samples for each time point and normalized to HARP mRNA expression using PCR. Taqman arbitrary values (left abscissa) represent the average of three samples for Th1-inducing (A) and Th2-inducing (B) conditions. Error bars represent the standard deviation. Microarray values (right abscissa) for each sample represent the average differences calculated using Affymetrix software.

FIG. 2 depicts a comparison of gene expression in Th1-inducing and Th2-inducing conditions. RNA from each treatment group was fluorescently labeled and hybridized to Affymetrix gene microarrays as described in the Exemplification. Average differences for each gene under Th1-inducing and Th2-inducing conditions were generated by normalizing both samples to a naïve CD4⁺ T cell baseline and were plotted against each other. Genes that were designated absent in all samples were deleted from the analysis. Genes with average differences less than or equal to 0 were defaulted to 1. Lines drawn on the group represent greater than 2 fold increase in Th1 compared to Th2-inducing conditions, or a greater than 2 fold increase in Th2 compared to Th1-inducing conditions.

FIGS. 3A and 3B depict differentiated Th1 and Th2 cell population phenotypes. Naïve CD4′ T cells isolated from cord blood were cultured for the indicated times in the presence of microbeads coated with anti-CD3 and anti-CD28 and 10 ng/ml rIL-2 for 7 days (Th0 cells) and either 10 ng/ml rhIL-12 and 200 ng/ml anti-Il-4 (Th1 cells) or 10 ng/ml rIL-4 and 2 microgram/ml anti-IL-12 (Th2 cells). Cell populations were then restimulated for 4 or 24 hours with (+) or without (−) PMA and ionomycin. IFN-γ (A) and IL-4 (B) production in the culture supernatant were assayed by ELISA. Results represent the mean and standard deviation of triplicate samples and are representative of several experiments.

FIG. 4 depicts a cluster analysis of gene expression in CD4⁺ T cells cultured for 24 hours in Th1-inducing or Th2-inducing conditions. Total RNA from each treatment group was fluorescently labeled and hybridized to Affymetrix gene microarrays. Gene expression was expressed as fold change over the naïve CD4⁺ T cell baseline, designated as 1. Fold changes of less than 2 were eliminated from the analysis, and the genes were then clustered hierarchically into groups on the basis of the similarity of their expression profiles. The expression pattern of each gene is represented by a horizontal line. The graphs on the right depict the average expression profiles of the corresponding “clusters” identified (A-H, indicated by bars on the right of the cluster diagram).

### DETAILED DESCRIPTION OF THE INVENTION

The present invention is obtained at least in part from a study of the expression of a large number of genes in human Th1 and Th2 cell populations generated in vitro. Gene expression during both the early stages of T helper cell differentiation and in restimulated Th1 and Th2 cell populations was compared to expression in undifferentiated T cell populations. Global gene expression analysis unexpectedly revealed that the number of genes differentially expressed under Th1 and Th2-inducing conditions is greater in the first 24 hours of differentiation as compared to gene expression patterns in fully differentiated Th1 and Th2 cell populations which have been restimulated. This finding indicates that differentiated populations of Th1 and Th2 cells are relatively similar at the molecular level, while a large number of genes may be involved in the early stages of Th1 and Th2 development. Additionally, differential gene expression was observed across a wide variety of different functional gene categories, (for example, genes involved in such diverse functions as protein degradation and chromatin remodeling). The markers of the invention, set forth in Tables 1-12, are linked to Th1 and/or Th2 cells during either the first 24 hours of differentiation into Th1 or Th2 cells (Tables 1-6) or during restimulation of fully differentiated Th1 or Th2 cells (Tables 7-11). These markers may be used according to the methods and compositions set forth below, to identify a Th1 or Th2 cell, to discriminate between Th1 and Th2 cell populations in a sample, or to selectively promote or inhibit Th1 or Th2 cell population growth.

Further, a number of diseases and conditions have been identified which are known to be associated with the activity and/or quantity of Th1 or Th2 cell populations in a subject. For example, inflammatory bowel disease (including Crohn's disease), multiple sclerosis, rheumatoid arthritis, and psoriasis (including psoriatic arthritis) have been linked to aberrant Th1 activity. Similarly, allergy and asthma have been linked to aberrant Th2 activity (Liblau et al. (1995) Immunol Today 1995 16(1):34-8). The markers of the invention may also be used to identify aberrant activity and/or quantities of Th1 and/or Th2 cells in a subject.

The present invention is based, at least in part, on the identification of a number of genetic markers, set forth in Tables 1-12, which are differentially expressed in Th1 and Th2 cells. A panel of 6800 known genes was screened for expression in naïve CD4⁺ cells which had been induced to differentiate into Th1 or Th2 cells. Those genes with statistically significant differences (e.g., at least two-fold difference) between expression in Th1 and/or Th2 cells, as compared to naïve, undifferentiated CD4⁺ T cells, are set forth in Tables 1-12. This differential expression was observed either as a decrease in expression (Tables 2, 4, and 9), or an increase in expression (Table 3, 5, 8, and 10).

Several markers were known prior to the invention to be associated specifically with either Th1 or Th2 cells. These markers are set forth in Table 12. These markers are not included with the markers of the invention. However, these markers may be conveniently used in combination with the markers of the invention in the methods, panels, and kits of the invention.

Gene expression in Th1 and Th2 cells was assessed under different induction conditions. In a first experiment, naïve CD4+T cells were induced to differentiate into either Th1 or Th2 cells by incubation with IL-12 or IL-4, respectively, for 24 hours (Experiment 1, Tables 1-6). In a second experiment, induction of differentiation by cytokine incubation was permitted to take place over seven days, after which time the differentiated T cells were stimulated with PMA and ionomycin (Experiment 2, Tables 7-1 1). In both experiments, total labeled RNA was prepared from the treated cell populations, and was hybridized to GeneChip arrays on which a panel of 6800 known genes is linked. Hybridization was quantified and compared to naïve T cell control values. Table 1 sets forth those genes which were differentially expressed (e.g., increased or decreased at least two-fold as compared to control values) in the first experiment. Table 2 includes those genes from Table 1 in which a gene was observed to have decreased expression in Th1 but unchanged expression in Th2. Table 3 includes those genes from Table 1 in which a gene was observed to have increased expression in Th1 but unchanged expression in Th2. Table 4 includes those genes from Table 1 in which a gene was observed to have unchanged expression in Th1 cells but decreased expression in Th2 cells. Table 5 includes those genes from Table 1 with unchanged expression in Th1 cells but increased expression in Th2 cells. Table 6 includes only those genes which have changed expression (either increased or decreased) in both Th1 and Th2 cells, as compared to naïve CD4⁺ T cells.

Those genes which were identified in Experiment 2 as being differentially expressed in mature, restimulated Th1 and Th2 cells are set forth in Table 7. Table 8 includes those genes from Table 7 which were increased in expression in Th1 cells and unchanged in expression in Th2 cells. Table 9 includes those genes from Table 7 which were decreased in expression in Th1 cells and increased in expression in Th2 cells. Table 10 includes those genes from Table 7 which were increased in Th2 cells but which were unchanged in expression in Th1 cells. Table 11 includes those genes from Table 7 which were changed in expression (e.g., increased or decreased) in both Th1 and Th2 cells relative to naïve T cell controls.

The present invention pertains to the use of the genes set forth in Tables 1-12 (e.g., the DNA or cDNA), the corresponding mRNA transcripts, and the encoded polypeptides as markers for the presence of a Th1 or Th2 cell. For example, the gene designated ‘TAP2’ (accession number M74447) is increased in expression level in Th1 cells relative to naïve T cells, but is unchanged in expression in Th2 cells (Table 3), and therefore serves as a marker for Th1 cells but not Th2 cells. Both the presence of increased or decreased mRNA for this gene (and/or for other genes set forth in Tables 1 -12), and also increased or decreased levels of the protein products of this gene (and/or other genes set forth in Tables 1-12) serve as markers of Th1 or Th2 cells. Panels of the markers can also be conveniently arrayed for use in kits or on solid supports.

Similarly, the present invention also pertains to the use of the genes set forth in Tables 1-12 (e.g., the DNA or cDNA), the corresponding mRNA transcripts, and the encoded polypeptides as markers for distinguishing the maturity of a Th1 or Th2 cell. Those markers in Table 7 which are not also included in Table 1 (e.g., all excepting IFNG, SCYA20, and APT1) serve as markers specific for already-differentiated and restimulated Th1 or Th2 cells, whereas those markers in Table 1 which are not also included in Table 7 (e.g., all excepting IFNG, SCYA20 and APT1) serve as markers specific for Th1 or Th2 cells in the first 24 hours of differentiation. Further, the markers included in Tables 2 and 3 (with the exception of IFNG, SCYA20, and APT1) are specific for newly differentiated Th1 cells, and the markers set forth in Tables 4 and 5 are specific for newly differentiated Th2 cells, whereas the markers set forth in Tables 8 and 9 (with the exception of IFNG and SCYA20) are specific for fully differentiated and restimulated Th1 cells and the markers included in Table 10 (with the exception of APT1) are specific for fully differentiated and restimulated Th2 cells. Panels of the markers can be conveniently arrayed for use in kits or on solid supports.

The present invention also pertains to the use of the genes set forth in Tables 1 -12 (e.g., the DNA or cDNA), the corresponding mRNA transcripts, and the encoded polypeptides as markers for the presence or risk of development of a Th1- or Th2-associated condition. These markers are further useful to correlate the extent and/or severity of disease. Panels of the markers can be conveniently arrayed for use in kits or on solid supports. The markers can also be useful in the treatment of a Th1- or Th2-associated condition, or in assessing the efficacy of a treatment for a Th1- or Th2-associated condition.

In another aspect, the invention provides markers whose quantity or activity is correlated with the presence of a Th1- or Th2-associated condition. The markers of the invention may be nucleic acid molecules (e.g., DNA, cDNA, or RNA) or polypeptides. These markers are either increased or decreased in quantity or activity in T cell samples from a diseased subject than in T cell samples from a normal subject. For example, the gene designated ‘TAP2’ (accession number M74447) is increased in expression level in Th1 cells relative to naïve T cells, but is unchanged in expression in Th2 cells (Table 3), and therefore may serve as a marker for a disease associated with Th1 cell population or activity (e.g., psoriasis or multiple sclerosis).

Preferably, increased and decreased levels of the markers of the invention are increases and decreases of a magnitude that is statistically significant as compared to appropriate control samples (e.g., naïve T cells, or samples not affected with a Th1- or Th2-associated condition). In particularly preferred embodiments, the marker is increased or decreased relative to control samples by at least 2-, 3-, 4-, 5-, 6-, 7-, 8-, 9-, or 10-fold or more. Similarly, one skilled in the art will be cognizant of the fact that a preferred detection methodology is one in which the resulting detection values are above the minimum detection limit of the methodology.

Measurement of the relative amount of an RNA or protein marker of the invention may be by any method known in the art (see, e.g, Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary nucleic acid molecule) specific for the target RNA to the extracted RNA, and detection of the probe (e.g., Northern blotting). Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and nucleic acid molecules may also be assessed by gel electrophoresis, column chromatography, direct sequencing, or quantitative PCR (in the case of nucleic acid molecules) among many other techniques well known to those skilled in the art.

In certain embodiments, the genes themselves (e.g, the DNA or CDNA) may serve as markers for Th1 or Th2 cells, or for a Th1- or Th2-associated condition. For example, the absence of nucleic acids corresponding to a gene (e.g., a gene from Table 1), such as by deletion of all or part of the gene, may be correlated with either a Th1 or Th2 cell, or with a condition associated specifically with either of these cell types. Similarly, an increase of nucleic acid corresponding to a gene (e.g, a gene from Tables 1-12), such as by duplication of the gene, may also be correlated with either Th1 or Th2 cells, or with a condition associated specifically with either of these cell types.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g, a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

The invention also encompasses nucleic acid and protein molecules which are structurally different from the molecules described above (e.g., which have a slightly altered nucleic acid or amino acid sequence), but which have the same properties as the molecules above (e.g, encoded amino acid sequence, or which are changed only in nonessential amino acid residues). Such molecules include allelic variants, and are described in greater detail in subsection I.

In another aspect, the invention provides markers whose quantity or activity is correlated with the quantity or activity of Th1 or Th2 cells. These markers are either increased or decreased in quantity or activity in a sample in a fashion that is either positively or negatively correlated with the quantity or activity of Th1 or Th2 cells in the sample. In yet another aspect, the invention provides markers whose quantity or activity is correlated with the probability of biased development of Th1 or Th2 cells in a subject. These markers are either increased or decreased in activity or quantity in direct correlation to the likelihood of the differentiation of naïve T cells into either Th1 or Th2 cells.

In another aspect, the invention provides markers whose quantity or activity is correlated with the severity of a Th1- or Th2-associated condition (see, e.g., Example 3). These markers are either increased or decreased in quantity or activity in a tissue affected by the Th1 or Th2-associated condition in a fashion that is either positively or negatively correlated with the degree of severity of the Th1- or Th2-associated condition. In yet another aspect, the invention provides markers whose quantity or activity is correlated with a risk in a subject for developing a Th1- or Th2-associated condition. These markers are either increased or decreased in activity or quantity in direct correlation to the likelihood of the development of a Th1- or Th2-associated condition in a subject.

Each marker may be considered individually, although it is within the scope of the invention to provide combinations of two or more markers for use in the methods and compositions of the invention to increase the confidence of the analysis. In another aspect, the invention provides panels of the markers of the invention. In a preferred embodiment, these panels of markers are selected such that the markers within any one panel share certain features. For example, the markers of a first panel may each exhibit an increase in quantity or activity in Th1 cells as compared to Th2 cells or naïve CD4+cells, whereas the markers of a second panel may each exhibit a decrease in quantity or activity in Th1 cells as compared to Th2 cells or naïve CD4⁺ cells. Panels of the markers of the invention are set forth in Tables 1-12. It will be apparent to one skilled in the art that the methods and compositions of the invention may be practiced with any one of the panels set forth in Tables 1-12, or any portion or combination thereof.

It will also be appreciated by one skilled in the art that the panels of markers of the invention may conveniently be provided on solid supports. For example, polynucleotides, such as mRNA, may be coupled to an array (e.g., a GeneChip array for hybridization analysis), to a resin (e.g., a resin which can be packed into a column for column chromatography), or a matrix (e.g., a nitrocellulose matrix for northern blot analysis). The immobilization of molecules complementary to the marker(s), either covalently or noncovalently, permits a discrete analysis of the presence or activity of each marker in a sample. In an array, for example, polynucleotides complementary to each member of a panel of markers may individually be attached to different, known locations on the array. The array may be hybridized with, for example, polynucleotides extracted from a skin cell sample from a subject. The hybridization of polynucleotides from the sample with the array at any location on the array can be detected, and thus the presence or quantity of the marker in the sample can be ascertained. In a preferred embodiment, a “GeneChip” array is employed (Affymetrix). Similarly, Western analyses may be performed on immobilized antibodies specific for different polypeptide markers hybridized to a protein sample from a subject.

It will also be apparent to one skilled in the art that the entire marker protein or nucleic acid molecule need not be conjugated to the support; a portion of the marker of sufficient length for detection purposes (e.g., for hybridization), for example, a portion of the marker which is 7, 10, 15, 20, 25, 30, 35, 40, 45, 50, 55, 60, 65, 70, 75, 100 or more nucleotides or amino acids in length may be sufficient for detection purposes.

The nucleic acid and protein markers of the invention may be isolated from any tissue or cell of a subject in which T cells are found. In a preferred embodiment, the tissue is blood, thymus, spleen, lymph, pus, or bone marrow. However, it will be apparent to one skilled in the art that T cells may be present as an infiltrate in many other tissues, and that such tissues may also serve as sources from which the markers of the invention may be isolated, or in which the presence, activity, and/or quantity of the markers of the invention may be assessed. The tissue samples containing one or more of the markers themselves may be useful in the methods of the invention, and one skilled in the art will be cognizant of the methods by which such samples may be conveniently obtained, stored, and/or preserved.

In another aspect, the invention provides methods of making an isolated hybridoma which produces an antibody useful for assessing the presence, relative amounts, stage of maturity, or likelihood of development of Th1 and/or Th2 cells, or for assessing whether a patient is afflicted with a Th1- or Th2-associated condition. In this method, a protein corresponding to a marker of the invention is isolated (e.g., by purification from a cell in which it is expressed or by transcription and translation of a nucleic acid encoding the protein in vivo or in vitro using known methods. A vertebrate, preferably a mammal such as a mouse, rat, rabbit, or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

The invention provides methods of identifying the presence of a Th1 and/or Th2 cell in a subject, or of monitoring the development of Th1 and/or Th2 cells in a subject. These methods involve isolating one or more samples (e.g., multiple samples taken over a period of time) from a subject (e.g., a sample containing T helper cells), detecting the presence, quantity, and/or activity of one or more markers of the invention (e.g., markers from Tables 1-12) in each of the samples relative to a second sample not containing T helper cells, or containing naïve T cells. The levels of markers in the two or more samples are compared. A significant (greater than two-fold) increase or decrease in one or more markers in the test sample indicates the presence of a Th1 and/or Th2 cell in a subject. By monitoring the increase or decrease in marker expression in a series of samples taken over time from the subject, it is further possible to monitor the increase or decrease in Th1 and/or Th2 cells over the time period in which the samples were taken from the subject.

The invention also provides methods of determining the potential for differentiation of naïve T cells into Th1 and/or Th2 cells in a subject. These methods involve isolating a sample from a subject (e.g., a sample containing T helper cells), detecting the presence, quantity, and/or activity of one or more markers of the invention (e.g., markers from Tables 1-12) relative to a second sample not containing T helper cells, or containing naïve T cells. The levels of markers in the two samples are compared, and a significant (greater than two-fold) increase or decrease in one or more markers in the test sample is an indicator of the likelihood of development of Th1 or Th2 cells from naïve T cells in the subject.

The invention also provides methods of determining the growth or maturity of Th1 and/or Th2 cells in a sample. These methods involve isolating a sample from a subject (e.g., a sample containing T helper cells), detecting the presence, quantity, and/or activity of one or more markers of the invention (e.g., markers, not including IFNG, SCYA20, or APT1, from Tables 1-5 or markers, not including IFNG, SCYA20, or APT1, from Tables 7-10) relative to a second sample not containing T helper cells, containing naïve T cells, or containing known mature, restimulated, or newly differentiated Th1 or Th2 cells. The levels of markers in the two samples are compared, and a significant (greater than two-fold) increase or decrease in one or more markers in the test sample is an indicator of the likelihood of development of Th1 or Th2 cells from naïve T cells in the subject. For example, a significant decrease in the expression of a marker from Table 9 in the test sample relative to the control sample is indicative of the presence of a fully developed and restimulated Th1 cell in the test sample.

The invention provides methods of diagnosing a Th1- or Th2-associated condition, or risk of developing a Th1- or Th2-associated condition in a subject. These methods involve isolating a sample from a subject (e.g., a sample containing T helper cells), detecting the presence, quantity, and/or activity of one or more markers of the invention in the sample relative to a second sample from a subject known not to have a Th1- or Th2-associated condition. The levels of markers in the two samples are compared, and a significant increase or decrease in one or more markers in the test sample indicates the presence or risk of presence of a Th1- or Th2-associated condition in the subject.

The invention also provides methods of assessing the severity of a Th1- or Th2-associated condition in a subject. These methods involve isolating a sample from a subject (e.g., a sample containing T helper cells), detecting the presence, quantity, and/or activity of one or more markers of the invention in the sample relative to a second sample from a subject known not to have a Th1- or Th2-associated condition. The levels of markers in the two samples are compared, and a significant increase or decrease in one or more markers in the test sample is correlated with the degree of severity of a Th1- or Th2-associated condition in the subject.

The invention also provides methods of inhibiting the differentiation of naïve T cells into Th1 or Th2 cells, or of selectively inhibiting the growth and development of Th1 and/or Th2 cells in a subject. These methods involve isolating a sample from a subject (e.g., a sample containing T helper cells), detecting the presence, quantity, and/or activity of one or more markers of the invention in the sample relative to a second control sample (e.g., a sample containing no T cells, or naïve T cells). The levels of markers in the two samples are compared, and significant increases or decreases in one or more markers in the test sample relative to the control sample are observed. For markers that are significantly decreased in expression or activity, the subject may be administered that expressed marker protein, or may be treated by the introduction of mRNA or DNA corresponding to the decreased marker (e.g., by gene therapy), to thereby increase the levels of the marker protein in the subject. For markers that are significantly increased in expression or activity, the subject may be administered mRNA or DNA antisense to the increased marker (e.g., by gene therapy), or may be administered antibodies specific for the marker protein, to thereby decrease the levels of the marker protein in the subject. In this manner, the differentiation of naïve T cells into Th1 or Th2 cells, or the growth and development of Th1 and/or Th2 cells may be inhibited in a subject.

The invention also provides methods of increasing the differentiation of naïve T cells into Th1 or Th2 cells, or of selectively increasing the growth and development of Th1 and/or Th2 cells in a subject. These methods involve isolating a sample from a subject (e.g., a sample containing T helper cells), detecting the presence, quantity, and/or activity of one or more markers of the invention in the sample relative to a second control sample (e.g., a sample containing no T cells, or naïve T cells). The levels of markers in the two samples are compared, and significant increases or decreases in one or more markers in the test sample relative to the control sample are observed. For markers that are significantly increased in expression or activity, the subject may be administered that expressed marker protein, or may be treated by the introduction of mRNA or DNA corresponding to the increased marker (e.g., by gene therapy), to thereby further increase the levels of the marker protein in the subject. For markers that are significantly decreased in expression or activity, the subject may be administered mRNA or DNA antisense to the decreased marker (e.g., by gene therapy), or may be administered antibodies specific for the marker protein, to thereby further decrease the levels of the marker protein in the subject. In this manner, the differentiation of naïve T cells into Th1 or Th2 cells, or the growth and development of Th1 and/or Th2 cells may be increased in a subject.

The invention also provides methods of treating (e.g., inhibiting) a Th1- or Th2-associated condition in a subject. These methods involve isolating a sample from a subject (e.g., a sample containing T helper cells), detecting the presence, quantity, and/or activity of one or more markers of the invention in the sample relative to a second sample from a subject known not to have a Th1- or Th2-associated condition. The levels of markers in the two samples are compared, and significant increases or decreases in one or more markers in the test sample relative to the control sample are observed. For markers that are significantly decreased in expression or activity, the subject may be administered that expressed marker protein, or may be treated by the introduction of mRNA or DNA corresponding to the decreased marker (e.g., by gene therapy), to thereby increase the levels of the marker protein in the subject. For markers that are significantly increased in expression or activity, the subject may be administered mRNA or DNA antisense to the increased marker (e.g., by gene therapy), or may be administered antibodies specific for the marker protein, to thereby decrease the levels of the marker protein in the subject. In this manner, the subject may be treated for a Th1- or Th2-associated condition.

The invention also provides methods of preventing the development of a Th1- or Th2-associated condition in a subject. These methods involve, for markers that are significantly decreased in expression or activity, the administration of that marker protein, or the introduction of mRNA or DNA corresponding to the decreased marker (e.g., by gene therapy), to thereby increase the levels of the marker protein in the subject. For markers that are significantly increased in expression or activity, the subject may be administered mRNA or DNA antisense to the increased marker (e.g., by gene therapy), or may be administered antibodies specific for the marker protein, to thereby decrease the levels of the marker protein in the subject. In this manner, the development of a Th1- or Th2-associated condition in a subject may be prevented.

The invention also provides methods of assessing a treatment or therapy for promoting Th1 and/or Th2 differentiation or growth in a subject. These methods involve isolating a sample from a subject (e.g., a sample containing T helper cells) who is undergoing a treatment or therapy; detecting the presence, quantity, and/or activity of one or more markers of the invention in the first sample relative to a second sample from a subject who is not undergoing the treatment or therapy, or to a sample from the subject prior to treatment. The levels of markers in the two samples are compared, and significant increases or decreases in one or more markers in the first sample relative to the other samples are observed, and correlated with the presence, or level of maturity of Th1 and/or Th2 cells in the sample. By assessing the change in the sample in number or maturity level of Th1 and/or Th2 cells, the ability of the treatment or therapy to stimulate the differentiation of naïve T cells into Th1 or Th2 cells, or to stimulate the growth and maturation of Th1 and/or Th2 cells is also determined.

The invention also provides methods of assessing a treatment or therapy for inhibiting Th1 and/or Th2 differentiation or growth in a subject. These methods involve isolating a sample from a subject (e.g., a sample containing T helper cells) who is undergoing a treatment or therapy; detecting the presence, quantity, and/or activity of one or more markers of the invention in the first sample relative to a second sample from a subject who is not undergoing the treatment or therapy, or to a sample from the subject prior to treatment. The levels of markers in the two samples are compared, and significant increases or decreases in one or more markers in the first sample relative to the other samples are observed, and correlated with the presence, or level of maturity of Th1 and/or Th2 cells in the sample. By assessing the change in the sample in number or maturity level of Th1 and/or Th2 cells, the ability of the treatment or therapy to inhibit the differentiation of naïve T cells into Th1 or Th2 cells, or to inhibit the growth and maturation of Th1 and/or Th2 cells is also determined.

The invention also provides methods of assessing a treatment or therapy for its ability to trigger Th1 and/or Th2 differentiation, growth, or maturation in a subject. These methods involve isolating a sample from a subject (e.g., a subject having a certain probability of Th1 and/or Th2 cell differentiation, growth, or maturation) who is undergoing a treatment or therapy; detecting the presence, quantity, and/or activity of one or more markers of the invention in the first sample relative to a second sample from a subject who is not undergoing the treatment or therapy, or to a sample from the subject prior to treatment. The levels of markers in the two samples are compared, and significant increases or decreases in one or more markers in the first sample relative to the other samples are observed, and correlated with the presence, or level of maturity of Th1 and/or Th2 cells in the sample. By assessing the change in the sample in number or maturity level of Th1 and/or Th2 cells, the ability of the treatment or therapy to trigger differentiation of naïve T cells into Th1 or Th2 cells, or to trigger the growth and maturation of Th1 and/or Th2 cells is also determined.

The invention also provides methods of assessing a treatment or therapy for a Th1- or Th2-associated condition in a subject. These methods involve isolating a sample from a subject (e.g., a sample containing T helper cells) suffering from a Th1- or Th2-associated condition who is undergoing a treatment or therapy, detecting the presence, quantity, and/or activity of one or more markers of the invention in the first sample relative to a second sample from a subject afflicted with a Th1- or Th2-associated condition who is not undergoing any treatment or therapy for the condition, and also relative to a third sample from a subject unafflicted by a Th1- or Th2-associated condition or from a tissue in the same subject known not to be affected by the presence of a Th1- or Th2-associated condition. The levels of markers in the three samples are compared, and significant increases or decreases in one or more markers in the first sample relative to the other samples are observed, and correlated with the presence, risk of presence, or severity of a Th1- or Th2-associated condition. By assessing whether a Th1- or Th2-associated condition has been lessened or alleviated in the sample, the ability of the treatment or therapy to treat a Th1- or Th2-associated condition is also determined.

The invention also provides pharmaceutical compositions for the stimulation of differentiation of naïve T cells into Th1 or Th2 cells, or for the stimulation of growth of Th1 or Th2 cells. These compositions may include a marker protein and/or nucleic acid of the invention (e.g., for those markers which are increased in quantity or activity in Th1 or Th2 cells versus naïve T cells), and can be formulated as described herein. Alternately, these compositions may include an antibody which specifically binds to an inhibitor of a marker protein of the invention, and can be formulated as described herein.

The invention also provides pharmaceutical compositions for the inhibition of differentiation of naïve T cells into Th1 or Th2 cells, or for the inhibition of growth of Th1 or Th2 cells. These compositions may include a marker protein and/or nucleic acid of the invention (e.g., for those markers which are decreased in quantity or activity in psoriatic tissue versus nonpsoriatic tissue), and can be formulated as described herein. Alternately, these compositions may include an antibody which specifically binds to a marker protein of the invention and/or an antisense nucleic acid molecule which is complementary to a marker nucleic acid of the invention (e.g., for those markers which are increased in quantity or activity in diseased tissue versus nondiseased tissue), and can be formulated as described herein.

The invention also provides pharmaceutical compositions for the treatment of a Th1- or Th2-associated condition. These compositions may include a marker protein and/or nucleic acid of the invention (e.g., for those markers which are decreased in quantity or activity in psoriatic tissue versus nonpsoriatic tissue), and can be formulated as described herein. Alternately, these compositions may include an antibody which specifically binds to a marker protein of the invention and/or an antisense nucleic acid molecule which is complementary to a marker nucleic acid of the invention (e.g., for those markers which are increased in quantity or activity in diseased tissue versus nondiseased tissue), and can be formulated as described herein.

The invention also provides kits for assessing the presence or likelihood of development of Th1 and/or Th2 cells and/or for assessing the presence of newly differentiated versus mature Th1 or Th2 cells in a sample (e.g., a sample from a subject), the kit comprising an antibody, wherein the antibody specifically binds with a protein corresponding to a marker selected from the group consisting of the markers listed in Tables 1-12.

The invention also provides kits for assessing the presence of cells participating in a Th1- or Th-2-associated condition in a sample (e.g., a sample from a subject at risk for a Th1- or Th2-associated condition), the kit comprising an antibody, wherein the antibody specifically binds with a protein corresponding to a marker selected from the group consisting of the markers listed in Tables 1-12.

The invention also provides kits for assessing the presence or likelihood of development of Th1 and/or Th2 cells and/or for assessing the presence of newly differentiated versus mature Th1 or Th2 cells in a sample (e.g., a sample from a subject), the kit comprising a nucleic acid probe wherein the probe specifically binds with a transcribed polynucleotide corresponding to a marker selected from the group consisting of the markers listed in Tables 1-12.

The invention further provides kits for assessing the presence of cells participating in a Th1- or Th2-associated condition in a sample from a subject (e.g., a subject at risk for a Th1- or Th2-associated condition), the kit comprising a nucleic acid probe wherein the probe specifically binds with a transcribed polynucleotide corresponding to a marker selected from the group consisting of the markers listed in Tables 1-12.

The invention further provides kits for assessing the suitability of each of a plurality of compounds for inhibiting the differentiation or growth of Th1 or Th2 cells in a subject. Such kits include a plurality of compounds to be tested, and a reagent for assessing expression of a marker selected from the group consisting of one or more of the markers set forth in Tables 1-12.

The invention further provides kits for assessing the suitability of each of a plurality of compounds for increasing the differentiation or growth of Th1 or Th2 cells in a subject. Such kits include a plurality of compounds to be tested, and a reagent for assessing expression of a marker selected from the group consisting of one or more of the markers set forth in Tables 1-12.

The invention further provides kits for assessing the suitability of each of a plurality of compounds for inhibiting a Th1- or Th2-associated condition in a subject. Such kits include a plurality of compounds to be tested, and a reagent for assessing expression of a marker selected from the group consisting of one or more of the markers set forth in Tables 1-12.

In another embodiment, the invention makes use of the genes set forth in Table 13 as markers associated specifically with Th1 or Th2 cells.

Modifications to the above-described compositions and methods of the invention, according to standard techniques, will be readily apparent to one skilled in the art and are meant to be encompassed by the invention.

To facilitate an understanding of the present invention, a number of terms and phrases are defined below:

As used herein, the term “modulation” includes, in its various grammatical forms (e.g., “modulated”, “modulation”, “modulating”, etc.), up-regulation, induction, stimulation, potentiation, and/or relief of inhibition, as well as inhibition and/or down-regulation.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. This, the term “polynucleotide sequence” is the alphabetical representation of a polynucleotide molecule. This alphabetical representation can be inputted into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “gene product” includes an amino acid (e.g., peptide or polypeptide) generated when a gene is transcribed and translated.

As used herein, a “polynucleotide corresponds to” another (a first) polynucleotide if it is related to the first polynucleotide by any of the following relationships:

1) The second polynucleotide comprises the first polynucleotide and the second polynucleotide encodes a gene product.

2) The second polynucleotide is 5′ or 3′ to the first polynucleotide in cDNA, RNA, genomic DNA, or fragments of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene coding for a gene product. The second polynucleotide may be an exon of the gene while the first polynucleotide may be an intron of the gene. 3) The second polynucleotide is the complement of the first polynucleotide.

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are well known in the art, and are taught, for example, in MacPherson et al. , IRL Press at Oxford University Press (1991)). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest.

A “gene delivery vehicle” includes a molecule that is capable of inserting one or more polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses and viral vectors, such as baculovirus, adenovirus, and retrovirus, bacteriophage, cosmid, plasmid, fungal vector and other recombination vehicles typically used in the art which have been described for replication and/or expression in a variety of eukaryotic and prokaryotic hosts. The gene delivery vehicles may be used for replication of the inserted polynucleotide, gene therapy as well as for simply polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above function.

A “host cell” is intended to include any individual cell or cell culture which can be or has been a recipient for vectors or for the incorporation of exogenous nucleic acid molecules, polynucleotides and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, e.g., murine, rat, simian or human cells.

The term “genetically modified” includes a cell containing and/or expressing a foreign gene or nucleic acid sequence which in turn modifies the genotype or phenotype of the cell or its progeny. This term includes any addition, deletion, or disruption to a cell's endogenous nucleotides.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Differentially expressed”, as applied to a gene, includes the differential production of mRNA transcribed from a gene or a protein product encoded by the gene. A differentially expressed gene may be overexpressed or underexpressed as compared to the expression level of a normal or control cell. In one aspect, it includes a differential that is 2.5 times, preferably 5 times or preferably 10 times higher or lower than the expression level detected in a control sample. The term “differentially expressed” also includes nucleotide sequences in a cell or tissue which are expressed where silent in a control cell or not expressed where expressed in a control cell.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprises an antigen recognition site of the required specificity.

As used herein, the term “Th1-associated condition” includes diseases and conditions in which T-helper 1 (Th1 ) cells are believed to play a role, in either the origin or progression of the disease. Examples of Th1-associated conditions include, but are not limited to, irritable bowel syndrome (including Crohn's disease), rheumatoid arthritis, multiple sclerosis, and psoriasis (see, e.g., Liblau et al. (1995) Immunol Today 1995 16(l):34-8).

As used herein, the term “Th2-associated condition” includes diseases and conditions in which T-helper 2 (Th2) cells are believed to play a role, in either the origin or progression of the disease. Examples of Th2-associated conditions include, but are not limited to, allergy and asthma (see, e.g., Liblau et al. (1995) Immunol Today 1995 16(1):34-8).

As used herein, the term “diseased tissue” includes a biological tissue from a subject afflicted with a Th1- or Th2-associated disease. As used herein, the term “nondiseased tissue” includes a biological tissue from a subject not afflicted with a Th1- or Th2-associated disease, or a tissue from a diseased individual which itself is not affected by the Th1- or Th2-associated disease. Preferred biological tissues are those including T helper cells, such as blood, serum, lymph, thymus, spleen, bone marrow, or pus.

As used herein, the term “marker” includes a polynucleotide or polypeptide molecule which is present or absent, or increased or decreased in quantity or activity in subjects afflicted with a Th1- or Th2-associated condition, or in cells involved in a Th1- or Th2-associated condition. The relative change in quantity or activity of the marker is correlated with the incidence or risk of incidence of a Th1- or Th2-associated condition.

As used herein, the term “panel of markers” includes a group of markers, the quantity or activity of each member of which is correlated with the incidence or risk of incidence of a Th1- or Th2-associated condition. In certain embodiments, a panel of markers may include only those markers which are either increased or decreased in quantity or activity in subjects afflicted with or cells involved in a Th1- or Th2-associated condition. In other embodiments, a panel of markers may include only those markers present in a specific tissue type which are correlated with the incidence or risk of incidence of a Th1- or Th2-associated condition.

Various aspects of the invention are described in further detail in the following subsections:

I. Isolated Nucleic Acid Molecules

One aspect of the invention pertains to isolated nucleic acid molecules that either themselves are the genetic markers (e.g., mRNA) of the invention, or which encode the polypeptide markers of the invention, or fragments thereof. Another aspect of the invention pertains to isolated nucleic acid fragments sufficient for use as hybridization probes to identify the nucleic acid molecules encoding the markers of the invention in a sample, as well as nucleotide fragments for use as PCR primers for the amplification or mutation of the nucleic acid molecules which encode the markers of the invention. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated marker nucleic acid molecule of the invention, or nucleic acid molecule encoding a polypeptide marker of the invention, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of one of the genes set forth in Tables 1-12, or a portion thereof, can be isolated using standard molecular biology techniques and the sequence information provided herein. Using all or portion of the nucleic acid sequence of one of the genes set forth in Tables 1-12 as a hybridization probe, a marker gene of the invention or a nucleic acid molecule encoding a polypeptide marker of the invention can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of a marker of the invention (e.g., a gene set forth in Tables 1-12), or a portion of any of these nucleotide sequences. A nucleic acid molecule which is complementary to such a nucleotide sequence is one which is sufficiently complementary to the nucleotide sequence such that it can hybridize to the nucleotide sequence, thereby forming a stable duplex.

The nucleic acid molecule of the invention, moreover, can comprise only a portion of the nucleic acid sequence of a marker nucleic acid of the invention, or a gene encoding a marker polypeptide of the invention, for example, a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of a marker nucleic acid, or a nucleic acid encoding a marker polypeptide of the invention.

Probes based on the nucleotide sequence of a marker gene or of a nucleic acid molecule encoding a marker polypeptide of the invention can be used to detect transcripts or genomic sequences corresponding to the marker gene(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a marker polypeptide of the invention, or which have greater or fewer copies of a marker gene of the invention. For example, a level of a marker polypeptide-encoding nucleic acid in a sample of cells from a subject may be detected, the amount of mRNA transcript of a gene encoding a marker polypeptide may be determined, or the presence of mutations or deletions of a marker gene of the invention may be assessed.

The invention further encompasses nucleic acid molecules that differ from the nucleic acid sequences of the genes set forth in Tables 1-12, due to degeneracy of the genetic code and which thus encode the same proteins as those encoded by the genes shown in Tables 1-12.

In addition to the nucleotide sequences of the genes set forth in Tables 1-12, it will be appreciated by those skilled in the art that DNA sequence polymorphisms that lead to changes in the amino acid sequences of the proteins encoded by the genes set forth in Tables 1-12 may exist within a population (e.g., the human population). Such genetic polymorphism in the genes set forth in Tables 1-12 may exist among individuals within a population due to natural allelic variation. An allele is one of a group of genes which occur alternatively at a given genetic locus. In addition it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus or to a polypeptide encoded by the nucleotide sequence. As used herein, the terms “gene” and “recombinant gene” refer to nucleic acid molecules which include an open reading frame encoding a marker polypeptide of the invention.

Nucleic acid molecules corresponding to natural allelic variants and homologues of the marker genes, or genes encoding the marker proteins of the invention can be isolated based on their homology to the genes set forth in Tables 1-12, using the cDNAs disclosed herein, or a portion thereof, as a hybridization probe according to standard hybridization techniques under stringent hybridization conditions. Nucleic acid molecules corresponding to natural allelic variants and homologues of the marker genes of the invention can further be isolated by mapping to the same chromosome or locus as the marker genes or genes encoding the marker proteins of the invention.

In another embodiment, an isolated nucleic acid molecule of the invention is at least 15, 20, 25, 30, 50, 100, 150, 200, 250, 300, 350, 400, 450, 500, 550, 600, 650, 700, 750, 800, 850, 900,950, 1000, 1100, 1200, 1300, 1400, 1500, 1600, 1700, 1800, 1900, 2000 or more nucleotides in length and hybridizes under stringent conditions to a nucleic acid molecule corresponding to a nucleotide sequence of a marker gene or gene encoding a marker protein of the invention. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of one of the genes set forth in Tables 1 -12 corresponds to a naturally-occurring nucleic acid molecule. As used herein, a “naturally-occurring” nucleic acid molecule includes an RNA or DNA molecule having a nucleotide sequence that occurs in nature (e.g., encodes a natural protein).

In addition to naturally-occurring allelic variants of the marker gene and gene encoding a marker protein of the invention sequences that may exist in the population, the skilled artisan will further appreciate that changes can be introduced by mutation into the nucleotide sequences of the marker genes or genes encoding the marker proteins of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to nucleic acid molecules encoding a marker protein of the invention that contain changes in amino acid residues that are not essential for activity. Such proteins differ in amino acid sequence from the marker proteins encoded by the genes set forth in Tables 1-12, yet retain biological activity. In one embodiment, the protein comprises an amino acid sequence at least about 60%, 65%, 70%, 75%, 80%, 85%, 90%, 95%, 98% or more homologous to a marker protein of the invention.

An isolated nucleic acid molecule encoding a protein homologous to a marker protein of the invention can be created by introducing one or more nucleotide substitutions, additions or deletions into the nucleotide sequence of the gene encoding the marker protein, such that one or more amino acid substitutions, additions or deletions are introduced into the encoded protein. Mutations can be introduced into the genes of the invention (e.g., a gene set forth in Tables 1-12) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of a coding sequence of a gene of the invention, such as by saturation mutagenesis, and the resultant mutants can be screened for biological activity to identify mutants that retain activity. Following mutagenesis, the encoded protein can be expressed recombinantly and the activity of the protein can be determined.

Another aspect of the invention pertains to isolated nucleic acid molecules which are antisense to the marker genes and genes encoding marker proteins of the invention. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand of a gene of the invention (e.g., a gene set forth in Tables 1-12), or to only a portion thereof. In one embodiment, an antisense nucleic acid molecule is antisense to a “coding region” of the coding strand of a nucleotide sequence of the invention. The term “coding region” includes the region of the nucleotide sequence comprising codons which are translated into amino acid. In another embodiment, the antisense nucleic acid molecule is antisense to a “noncoding region” of the coding strand of a nucleotide sequence of the invention. The term “noncoding region” includes 5′ and 3′ sequences which flank the coding region that are not translated into amino acids (i.e., also referred to as 5′ and 3′ untranslated regions).

Antisense nucleic acids of the invention can be designed according to the rules of Watson and Crick base pairing. The antisense nucleic acid molecule can be complementary to the entire coding region of an mRNA corresponding to a gene of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a marker protein of the invention to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site (e.g., in skin). Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts of the genes of the invention (e.g., a gene set forth in Tables 1-12) to thereby inhibit translation of this mRNA. A ribozyme having specificity for a marker protein-encoding nucleic acid can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a marker protein-encoding mRNA. See, e.g., Cech et al. U.S. Pat. No. 4,987,071; and Cech et al. U.S. Pat. No. 5,116,742. Alternatively, mRNA transcribed from a gene of the invention can be used to select a catalytic RNA having a specific ribonuclease activity from a pool of RNA molecules. See, e.g., Bartel, D. and Szostak, J. W. (1993) Science 261:1411-1418.

Alternatively, expression of a gene of the invention (e.g., a gene set forth in Tables 1-12) can be inhibited by targeting nucleotide sequences complementary to the regulatory region of these genes (e.g., the promoter and/or enhancers) to form triple helical structures that prevent transcription of the gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs can be used in therapeutic and diagnostic applications. For example, PNAs can be used as antisense or antigene agents for sequence-specific modulation of gene expression by, for example, inducing transcription or translation arrest or inhibiting replication. PNAs of the nucleic acid molecules of the invention (e.g., a gene set forth in Tables 1-12) can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the nucleic acid molecules of the invention can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label (e.g., a molecular beacon, as described in U.S. Pat. No. 5,876,930.

II. Isolated Proteins and Antibodies

One aspect of the invention pertains to isolated marker proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, marker proteins are produced by recombinant DNA techniques. Alternative to recombinant expression, a marker protein or polypeptide can be synthesized chemically using standard peptide synthesis techniques.

An “isolated” or “purified” protein or biologically active portion thereof is substantially free of cellular material or other contaminating proteins from the cell or tissue source from which the marker protein is derived, or substantially free from chemical precursors or other chemicals when chemically synthesized. The language “substantially free of cellular material” includes preparations of marker protein in which the protein is separated from cellular components of the cells from which it is isolated or recombinantly produced. In one embodiment, the language “substantially free of cellular material” includes preparations of marker protein having less than about 30% (by dry weight) of non-marker protein (also referred to herein as a “contaminating protein”), more preferably less than about 20% of non-marker protein, still more preferably less than about 10% of non-marker protein, and most preferably less than about 5% non-marker protein. When the marker protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of marker protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or non-protein chemicals, more preferably less than about 20% chemical precursors or non-protein chemicals, still more preferably less than about 10% chemical precursors or non-protein chemicals, and most preferably less than about 5% chemical precursors or non-protein chemicals.

As used herein, a “biologically active portion” of a marker protein includes a fragment of a marker protein comprising amino acid sequences sufficiently homologous to or derived from the amino acid sequence of the marker protein, which include fewer amino acids than the full length marker proteins, and exhibit at least one activity of a marker protein. Typically, biologically active portions comprise a domain or motif with at least one activity of the marker protein. A biologically active portion of a marker protein can be a polypeptide which is, for example, 10, 25, 50, 100, 200 or more amino acids in length. Biologically active portions of a marker protein can be used as targets for developing agents which modulate a marker protein-mediated activity.

In a preferred embodiment, marker protein is encoded by a gene set forth in Tables 1-12. In other embodiments, the marker protein is substantially homologous to a marker protein encoded by a gene set forth in Tables 1-12, and retains the functional activity of the marker protein, yet differs in amino acid sequence due to natural allelic variation or mutagenesis, as described in detail in subsection I above. Accordingly, in another embodiment, the marker protein is a protein which comprises an amino acid sequence at least about 60%, 65%, 70%, 75%, 80%, 85%, 90%, 95%, 98% or more homologous to the amino acid sequence encoded by a gene set forth in Tables 1 -12.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to marker protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides chimeric or fusion marker proteins. As used herein, a marker “chimeric protein” or “fusion protein” comprises a marker polypeptide operatively linked to a non-marker polypeptide. An “marker polypeptide” includes a polypeptide having an amino acid sequence encoded by a gene set forth in Tables 1-12, whereas a “non-marker polypeptide” includes a polypeptide having an amino acid sequence corresponding to a protein which is not substantially homologous to the marker protein, e.g., a protein which is different from marker protein and which is derived from the same or a different organism. Within a marker fusion protein the polypeptide can correspond to all or a portion of a marker protein. In a preferred embodiment, a marker fusion protein comprises at least one biologically active portion of a marker protein. Within the fusion protein, the term “operatively linked” is intended to indicate that the marker polypeptide and the non-marker polypeptide are fused in-frame to each other. The non-marker polypeptide can be fused to the N-terminus or C-terminus of the marker polypeptide.

For example, in one embodiment, the fusion protein is a GST-marker fusion protein in which the marker sequences are fused to the C-terminus of the GST sequences. Such fusion proteins can facilitate the purification of recombinant marker proteins.

In another embodiment, the fusion protein is a marker protein containing a heterologous signal sequence at its N-terminus. In certain host cells (e.g., mammalian host cells), expression and/or secretion of marker proteins can be increased through use of a heterologous signal sequence. Such signal sequences are well known in the art.

The marker fusion proteins of the invention can be incorporated into pharmaceutical compositions and administered to a subject in vivo, as described herein. The marker fusion proteins can be used to affect the bioavailability of a marker protein substrate. Marker fusion proteins may be useful therapeutically for the treatment of disorders (e.g., a Th1- or Th2-associated condition) caused by, for example, (i) aberrant modification or mutation of a gene encoding a marker protein; (ii) mis-regulation of the marker protein-encoding gene; and (iii) aberrant post-translational modification of a marker protein.

Moreover, the marker-fusion proteins of the invention can be used as immunogens to produce anti-marker protein antibodies in a subject, to purify marker protein ligands and in screening assays to identify molecules which inhibit the interaction of a marker protein with a marker protein substrate.

Preferably, a marker chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al John Wiley & Sons: 1992). Moreover, many expression vectors are commercially available that already encode a fusion moiety (e.g., a GST polypeptide). A marker protein-encoding nucleic acid can be cloned into such an expression vector such that the fusion moiety is linked in-frame to the marker protein.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the marker proteins of the invention which function as either agonists (mimetics) or as antagonists to the marker proteins. Variants of the marker proteins can be generated by mutagenesis, e.g., discrete point mutation or truncation of a marker protein. An agonist of the marker proteins can retain substantially the same, or a subset, of the biological activities of the naturally occurring form of a marker protein. An antagonist of a marker protein can inhibit one or more of the activities of the naturally occurring form of the marker protein by, for example, competitively modulating an activity of a marker protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the marker protein.

Variants of a marker protein which function as either marker protein agonists (mimetics) or as marker protein antagonists can be identified by screening combinatorial libraries of mutants, e.g., truncation mutants, of a marker protein for marker protein agonist or antagonist activity. In one embodiment, a variegated library of marker protein variants is generated by combinatorial mutagenesis at the nucleic acid level and is encoded by a variegated gene library. A variegated library of marker protein variants can be produced by, for example, enzymatically ligating a mixture of synthetic oligonucleotides into gene sequences such that a degenerate set of potential marker protein sequences is expressible as individual polypeptides, or alternatively, as a set of larger fusion proteins (e.g., for phage display) containing the set of marker protein sequences therein. There are a variety of methods which can be used to produce libraries of potential marker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential marker protein sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakuraetal. (1984) Science 198:1056; Ike et al. (1983) Nucleic Acid Res. 11:477).

In addition, libraries of fragments of a protein coding sequence corresponding to a marker protein of the invention can be used to generate a variegated population of marker protein fragments for screening and subsequent selection of variants of a marker protein. In one embodiment, a library of coding sequence fragments can be generated by treating a double stranded PCR fragment of a marker protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the marker protein.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify marker variants (Arkin and Yourvan (1992) Proc. Natl. Acad. Sci. USA 89:7811-7815; Delgrave et al. (1993) Protein Engineering 6(3):327-331).

An isolated marker protein, or a portion or fragment thereof, can be used as an immunogen to generate antibodies that bind marker proteins using standard techniques for polyclonal and monoclonal antibody preparation. A full-length marker protein can be used or, alternatively, the invention provides antigenic peptide fragments of these proteins for use as immunogens. The antigenic peptide of a marker protein comprises at least 8 amino acid residues of an amino acid sequence encoded by a gene set forth in Tables 1-12, and encompasses an epitope of a marker protein such that an antibody raised against the peptide forms a specific immune complex with the marker protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of the marker protein that are located on the surface of the protein, e.g., hydrophilic regions, as well as regions with high antigenicity.

A marker protein immunogen typically is used to prepare antibodies by immunizing a suitable subject, (e.g., rabbit, goat, mouse or other mammal) with the immunogen. An appropriate immunogenic preparation can contain, for example, recombinantly expressed marker protein or a chemically synthesized marker polypeptide. The preparation can further include an adjuvant, such as Freund's complete or incomplete adjuvant, or similar immunostimulatory agent. Immunization of a suitable subject with an immunogenic marker protein preparation induces a polyclonal anti-marker protein antibody response.

Accordingly, another aspect of the invention pertains to anti-marker protein antibodies. The term “antibody” as used herein includes immunoglobulin molecules and immunologically active portions of immunoglobulin molecules, i.e., molecules that contain an antigen binding site which specifically binds (immunoreacts with) an antigen, such as a marker protein. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to marker proteins. The term “monoclonal antibody” or “monoclonal antibody composition”, as used herein, includes a population of antibody molecules that contain only one species of an antigen binding site capable of immunoreacting with a particular epitope. A monoclonal antibody composition thus typically displays a single binding affinity for a particular marker protein with which it immunoreacts.

Polyclonal anti-marker protein antibodies can be prepared as described above by immunizing a suitable subject with a marker protein of the invention. The anti-marker protein antibody titer in the immunized subject can be monitored over time by standard techniques, such as with an enzyme linked immunosorbent assay (ELISA) using immobilized marker protein. If desired, the antibody molecules directed against marker proteins can be isolated from the mammal (e.g., from the blood) and further purified by well known techniques, such as protein A chromatography, to obtain the IgG fraction. At an appropriate time after immunization, e.g., when the anti-marker protein antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a marker protein immunogen as described above, and the culture supernatants of the resulting hybridoma cells are screened to identify a hybridoma producing a monoclonal antibody that binds to a marker protein of the invention.

Any of the many well known protocols used for fusing lymphocytes and immortalized cell lines can be applied for the purpose of generating an anti-marker protein monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind to a marker protein, e.g., using a standard ELISA assay.

Alternative to preparing monoclonal antibody-secreting hybridomas, a monoclonal anti-marker protein antibody can be identified and isolated by screening a recombinant combinatorial immunoglobulin library (e.g., an antibody phage display library) with marker protein to thereby isolate immunoglobulin library members that bind to a marker protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al (1992) Hum. Antibod. Hybridomas 3:81-85; Huse et al. (1989) Science 246:1275-1281; Griffiths et al. (1993) EMBO J 12:725-734; Hawkins et al (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-marker protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw etaL. (1988) J. Natl. Cancer Inst. 80:1553-1559); Morrison, S. L. (1985) Science 229:1202-1207; Oi et al. (1986) BioTechniques 4:214; Winter U.S. Patent 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An anti-marker protein antibody (e.g., monoclonal antibody) can be used to isolate a marker protein of the invention by standard techniques, such as affinity chromatography or immunoprecipitation. An anti-marker protein antibody can facilitate the purification of natural marker proteins from cells and of recombinantly produced marker proteins expressed in host cells. Moreover, an anti-marker protein antibody can be used to detect marker protein (e.g., in a cellular lysate or cell supernatant) in order to evaluate the abundance and pattern of expression of the marker protein. Anti-marker protein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I ¹³¹I ³⁵S or³H.

III. Recombinant Expression Vectors and Host Cells

Another aspect of the invention pertains to vectors, preferably expression vectors, containing a nucleic acid encoding a marker protein of the invention (or a portion thereof). As used herein, the term “vector” includes a nucleic acid molecule capable of transporting another nucleic acid to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., marker proteins, mutant forms of marker proteins, fusion proteins, and the like).

The recombinant expression vectors of the invention can be designed for expression of marker proteins in prokaryotic or eukaryotic cells. For example, marker proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRITS (Pharmacia, Piscataway, NJ) which fuse glutathione S-transferase (GST), maltose E binding protein, or protein A, respectively, to the target recombinant protein.

Purified fusion proteins can be utilized in marker activity assays, (e.g., direct assays or competitive assays described in detail below), or to generate antibodies specific for marker proteins, for example.

Examples of suitable inducible non-fusion E. coli expression vectors include pTrc (Amann et al., (1988) Gene 69:301-315) and pET 11d (Studier et al, Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. Coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the marker protein expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, marker proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a gene of the invention (e.g., a gene set forth in Tables 1-12). Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews-Trends in Genetics, Vol. 1(1) 1986.

Another aspect of the invention pertains to host celfs into which a nucleic acid molecule of the invention is introduced, e.g., a gene set forth in Tables 1-12 within a recombinant expression vector or a nucleic acid molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a marker protein of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a marker protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i. e., express) a marker protein. Accordingly, the invention further provides methods for producing a marker protein using the host cells of the invention. In one embodiment, the method comprises culturing the host cell of invention (into which a recombinant expression vector encoding a marker protein has been introduced) in a suitable medium such that a marker protein of the invention is produced. In another embodiment, the method further comprises isolating a marker protein from the medium or the host cell.

The host cells of the invention can also be used to produce non-human transgenic animals. For example, in one embodiment, a host cell of the invention is a fertilized oocyte or an embryonic stem cell into which marker-protein-coding sequences have been introduced. Such host cells can then be used to create non-human transgenic animals in which exogenous sequences encoding a marker protein of the invention have been introduced into their genome or homologous recombinant animals in which endogenous sequences encoding the marker proteins of the invention have been altered. Such animals are useful for studying the function and/or activity of a marker protein and for identifying and/or evaluating modulators of marker protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene of the invention (e.g., a gene set forth in Tables 1-12) has been altered by homologous recombination between the endogenous gene and an exogenous DNA molecule introduced into a cell of the animal, e.g., an embryonic cell of the animal, prior to development of the animal.

A transgenic animal of the invention can be created by introducing a marker-encoding nucleic acid into the male pronuclei of a fertilized oocyte, e.g, by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of a marker protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding a marker protein can further be bred to other transgenic animals carrying other transgenes.

To create a homologous recombinant animal, a vector is prepared which contains at least a portion of a gene of the invention into which a deletion, addition or substitution has been introduced to thereby alter, e.g., functionally disrupt, the gene. The gene can be a human gene, but more preferably, is a non-human homologue of a human gene of the invention (e.g., a gene set forth in Tables 1-12). For example, a mouse gene can be used to construct a homologous recombination nucleic acid molecule, e.g., a vector, suitable for altering an endogenous gene of the invention in the mouse genome. In a preferred embodiment, the homologous recombination nucleic acid molecule is designed such that, upon homologous recombination, the endogenous gene of the invention is functionally disrupted (i.e., no longer encodes a functional protein; also referred to as a “knock out” vector). Alternatively, the homologous recombination nucleic acid molecule can be designed such that, upon homologous recombination, the endogenous gene is mutated or otherwise altered but still encodes functional protein (e.g., the upstream regulatory region can be altered to thereby alter the expression of the endogenous marker protein). In the homologous recombination nucleic acid molecule, the altered portion of the gene of the invention is flanked at its 5′ and 3′ ends by additional nucleic acid sequence of the gene of the invention to allow for homologous recombination to occur between the exogenous gene carried by the homologous recombination nucleic acid molecule and an endogenous gene in a cell, e.g., an embryonic stem cell. The additional flanking nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g, Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced gene has homologously recombined with the endogenous gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g, by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The nucleic acid molecules of the invention (e.g., the genes set forth in Tables 1-12), fragments of marker proteins, and anti-marker protein antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker protein or an anti-marker protein antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Computer Readable Means and Arrays

Computer readable media comprising a marker(s) of the present invention is also provided. As used herein, “computer readable media” includes a medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a marker of the present invention.

As used herein, “recorded” includes a process for storing information on computer readable medium. Those skilled in the art can readily adopt any of the presently known methods for recording information on computer readable medium to generate manufactures comprising the markers of the present invention.

A variety of data processor programs and formats can be used to store the marker information of the present invention on computer readable medium. For example, the nucleic acid sequence corresponding to the markers can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the markers of the present invention.

By providing the markers of the invention in computer readable form, one can routinely access the marker sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

The invention also includes an array comprising a marker(s) of the present invention. The array can be used to assay expression of one or more genes in the array.

In one embodiment, the array can be used to assay gene expression in a tissue to ascertain tissue specificity of genes in the array. In this manner, up to about 8600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development and differentiation, disease progression, in vitro processes, such a cellular transformation and senescence, autonomic neural and neurological processes, such as, for example, pain and appetite, and cognitive functions, such as learning or memory.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and diseased cells. This provides a battery of genes that could serve as a molecular target for diagnosis or therapeutic intervention.

VI. Predictive Medicine

The present invention pertains to the field of predictive medicine in which diagnostic assays, prognostic assays, pharmacogenetics and monitoring clinical trials are used for prognostic (predictive) purposes to thereby treat an individual prophylactically. Accordingly, one aspect of the present invention relates to diagnostic assays for determining marker protein and/or nucleic acid expression as well as marker protein activity, in the context of a biological sample (e.g., blood, serum, cells, tissue) to thereby determine whether an individual is afflicted with a disease or disorder, or is at risk of developing a disorder, associated with increased or decreased marker protein expression or activity. The invention also provides for prognostic (or predictive) assays for determining whether an individual is at risk of developing a disorder associated with marker protein, nucleic acid expression or activity. For example, the number of copies of a marker gene can be assayed in a biological sample. Such assays can be used for prognostic or predictive purposes to thereby prophylactically treat an individual prior to the onset of a disorder (e.g., a Th1- or Th2-associated condition) characterized by or associated with marker protein, nucleic acid expression or activity.

Another aspect of the invention pertains to monitoring the influence of agents (e.g., drugs, compounds) on the expression or activity of marker in clinical trials.

These and other agents are described in further detail in the following sections.

1. Diagnostic Assays

An exemplary method for detecting the presence or absence of marker protein or nucleic acid of the invention in a biological sample involves obtaining a biological sample from a test subject and contacting the biological sample with a compound or an agent capable of detecting the protein or nucleic acid (e.g., mRNA, genomic DNA) that encodes the marker protein such that the presence of the marker protein or nucleic acid is detected in the biological sample. A preferred agent for detecting mRNA or genomic DNA corresponding to a marker gene or protein of the invention is a labeled nucleic acid probe capable of hybridizing to a mRNA or genomic DNA of the invention. Suitable probes for use in the diagnostic assays of the invention are described herein.

A preferred agent for detecting marker protein is an antibody capable of binding to marker protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect marker mRNA, protein, or genomic DNA in a biological sample in vitro as well as in vivo. For example, in vitro techniques for detection of marker mRNA include Northern hybridizations and in situ hybridizations. In vitro techniques for detection of marker protein include enzyme linked immunosorbent assays (ELISAs), Western blots, immunoprecipitations and immunofluorescence. In vitro techniques for detection of marker genomic DNA include Southern hybridizations. Furthermore, in vivo techniques for detection of marker protein include introducing into a subject a labeled anti-marker antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample (e.g., nondiseased tissue) from a control subject, contacting the control sample with a compound or agent capable of detecting marker protein, mRNA, or genomic DNA, such that the presence of marker protein, mRNA or genomic DNA is detected in the biological sample, and comparing the presence of marker protein, mRNA or genomic DNA in the control sample with the presence of marker protein, mRNA or genomic DNA in the test sample.

The invention also encompasses kits for detecting the presence of marker in a biological sample. For example, the kit can comprise a labeled compound or agent capable of detecting marker protein or mRNA in a biological sample; means for determining the amount of marker in the sample; and means for comparing the amount of marker in the sample with a standard. The compound or agent can be packaged in a suitable container. The kit can further comprise instructions for using the kit to detect marker protein or nucleic acid.

2. Prognostic Assays

The diagnostic methods described herein can furthermore be utilized to identify subjects having or at risk of developing a disease or disorder associated with aberrant marker expression or activity. As used herein, the term “aberrant” includes a marker expression or activity which deviates from the wild type marker expression or activity. Aberrant expression or activity includes increased or decreased expression or activity, as well as expression or activity which does not follow the wild type developmental pattern of expression or the subcellular pattern of expression. For example, aberrant marker expression or activity is intended to include the cases in which a mutation in the marker gene causes the marker gene to be under-expressed or over-expressed and situations in which such mutations result in a non-functional marker protein or a protein which does not function in a wild-type fashion, e.g., a protein which does not interact with a marker ligand or one which interacts with a non-marker protein ligand.

The assays described herein, such as the preceding diagnostic assays or the following assays, can be utilized to identify a subject having or at risk of developing a disorder associated with a misregulation in marker protein activity or nucleic acid expression, such as a Th1- or Th2-associated condition. Alternatively, the prognostic assays can be utilized to identify a subject having or at risk for developing a disorder associated with a misregulation in marker protein activity or nucleic acid expression, such as a Th1- or Th2-associated condition. Thus, the present invention provides a method for identifying a disease or disorder associated with aberrant marker expression or activity in which a test sample is obtained from a subject and marker protein or nucleic acid (e.g., mRNA or genomic DNA) is detected, wherein the presence of marker protein or nucleic acid is diagnostic for a subject having or at risk of developing a disease or disorder associated with aberrant marker expression or activity. As used herein, a “test sample” includes a biological sample obtained from a subject of interest. For example, a test sample can be a biological fluid (e.g., blood), cell sample, or tissue (e.g., skin).

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with increased or decreased marker expression or activity. For example, such methods can be used to determine whether a subject can be effectively treated with an agent for a disorder such as a Th1- or Th2-associated condition. Thus, the present invention provides methods for determining whether a subject can be effectively treated with an agent for a disorder associated with increased or decreased marker expression or activity in which a test sample is obtained and marker protein or nucleic acid expression or activity is detected (e.g., wherein the abundance of marker protein or nucleic acid expression or activity is diagnostic for a subject that can be administered the agent to treat a disorder associated with increased or decreased marker expression or activity).

The methods of the invention can also be used to detect genetic alterations in a marker gene, thereby determining if a subject with the altered gene is at risk for a disorder characterized by misregulation in marker protein activity or nucleic acid expression, such as a Th1- or Th2-associated condition. In preferred embodiments, the methods include detecting, in a sample of cells from the subject, the presence or absence of a genetic alteration characterized by at least one of an alteration affecting the integrity of a gene encoding a marker-protein, or the mis-expression of the marker gene. For example, such genetic alterations can be detected by ascertaining the existence of at least one of 1) a deletion of one or more nucleotides from a marker gene; 2) an addition of one or more nucleotides to a marker gene; 3) a substitution of one or more nucleotides of a marker gene, 4) a chromosomal rearrangement of a marker gene; 5) an alteration in the level of a messenger RNA transcript of a marker gene, 6) aberrant modification of a marker gene, such as of the methylation pattern of the genomic DNA, 7) the presence of a non-wild type splicing pattern of a messenger RNA transcript of a marker gene, 8) a non-wild type level of a marker-protein, 9) allelic loss of a marker gene, and 10) inappropriate post-translational modification of a marker-protein. As described herein, there are a large number of assays known in the art which can be used for detecting alterations in a marker gene. A preferred biological sample is a tissue (e.g., skin) or blood sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1 994) Proc. Natl. Acad. Sci. USA 91 :360-364), the latter of which can be particularly useful for detecting point mutations in the marker-gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a marker gene under conditions such that hybridization and amplification of the marker-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a marker gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in a marker gene or a gene encoding a marker protein of the invention can be identified by hybridizing a sample and control nucleic acids, e.g, DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in marker can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the marker gene and detect mutations by comparing the sequence of the sample marker with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the marker gene or gene encoding a marker protein of the invention include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes or genes encoding a marker protein of the invention. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993)Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose subjects exhibiting symptoms or family history of a disease or illness involving a marker gene.

Furthermore, any cell type or tissue in which marker is expressed may be utilized in the prognostic assays described herein.

3. Monitoring of Effects During Clinical Trials

Monitoring the influence of agents (e.g., drugs) on the expression or activity of a marker protein (e.g., the modulation of a Th1- or Th2-associated condition) can be applied not only in basic drug screening, but also in clinical trials. For example, the effectiveness of an agent determined by a screening assay as described herein to increase marker gene expression, protein levels, or upregulate marker activity, can be monitored in clinical trials of subjects exhibiting decreased marker gene expression, protein levels, or downregulated marker activity. Alternatively, the effectiveness of an agent determined by a screening assay to decrease marker gene expression, protein levels, or downregulate marker activity, can be monitored in clinical trials of subjects exhibiting increased marker gene expression, protein levels, or upregulated marker activity. In such clinical trials, the expression or activity of a marker gene, and preferably, other genes that have been implicated in, for example, a marker-associated disorder (e.g., a Th1- or Th2-associated condition) can be used as a “read out” or markers of the phenotype of a particular cell.

For example, and not by way of limitation, genes, including marker genes and genes encoding a marker protein of the invention, that are modulated in cells by treatment with an agent (e.g., compound, drug or small molecule) which modulates marker activity (e.g., identified in a screening assay as described herein) can be identified. Thus, to study the effect of agents on marker-associated disorders (e.g., a Th1- or Th2-associated condition), for example, in a clinical trial, cells can be isolated and RNA prepared and analyzed for the levels of expression of marker and other genes implicated in the marker-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of marker or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a marker protein, mRNA, or genomic DNA in the preadministration sample; (iii) obtaining one or more post-administration samples from the subject; (iv) detecting the level of expression or activity of the marker protein, mRNA, or genomic DNA in the post-administration samples; (v) comparing the level of expression or activity of the marker protein, mRNA, or genomic DNA in the pre-administration sample with the marker protein, mRNA, or genomic DNA in the post administration sample or samples; and (vi) altering the administration of the agent to the subject accordingly. For example, increased administration of the agent may be desirable to increase the expression or activity of marker to higher levels than detected, i.e., to increase the effectiveness of the agent. Alternatively, decreased administration of the agent may be desirable to decrease expression or activity of marker to lower levels than detected, i.e. to decrease the effectiveness of the agent. According to such an embodiment, marker expression or activity may be used as an indicator of the effectiveness of an agent, even in the absence of an observable phenotypic response.

C. Methods of Treatment

The present invention provides for both prophylactic and therapeutic methods of treating a subject at risk for (or susceptible to) a disorder or having a disorder associated with aberrant marker expression or activity. With regards to both prophylactic and therapeutic methods of treatment, such treatments may be specifically tailored or modified, based on knowledge obtained from the field of pharmacogenomics. “Pharmacogenomics”, as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype”, or “drug response genotype”.) Thus, another aspect of the invention provides methods for tailoring an individual's prophylactic or therapeutic treatment with either the marker molecules of the present invention or marker modulators according to that individual's drug response genotype. Pharmacogenomics allows a clinician or physician to target prophylactic or therapeutic treatments to subjects who will most benefit from the treatment and to avoid treatment of subjects who will experience toxic drug-related side effects.

1. Prophylactic Methods

In one aspect, the invention provides a method for preventing in a subject, a disease or condition (e.g., a Th1- or Th2-associated condition) associated with increased or decreased marker expression or activity, by administering to the subject a marker protein or an agent which modulates marker protein expression or at least one marker protein activity. Subjects at risk for a disease which is caused or contributed to by increased or decreased marker expression or activity can be identified by, for example, any or a combination of diagnostic or prognostic assays as described herein. Administration of a prophylactic agent can occur prior to the manifestation of symptoms characteristic of the differential marker protein expression, such that a disease or disorder is prevented or, alternatively, delayed in its progression. Depending on the type of marker aberrancy (e.g., increase or decrease in expression level), for example, a marker protein, marker protein agonist or marker protein antagonist agent can be used for treating the subject. The appropriate agent can be determined based on screening assays described herein.

2. Therapeutic Methods

Another aspect of the invention pertains to methods of modulating marker protein expression or activity for therapeutic purposes. Accordingly, in an exemplary embodiment, the modulatory method of the invention involves contacting a cell with a marker protein or agent that modulates one or more of the activities of a marker protein activity associated with the cell. An agent that modulates marker protein activity can be an agent as described herein, such as a nucleic acid or a protein, a naturally-occurring target molecule of a marker protein (e.g., a marker protein substrate), a marker protein antibody, a marker protein agonist or antagonist, a peptidomimetic of a marker protein agonist or antagonist, or other small molecule. In one embodiment, the agent stimulates one or more marker protein activities. Examples of such stimulatory agents include active marker protein and a nucleic acid molecule encoding marker protein that has been introduced into the cell. In another embodiment, the agent inhibits one or more marker protein activities. Examples of such inhibitory agents include antisense marker protein nucleic acid molecules, anti-marker protein antibodies, and marker protein inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a marker protein or nucleic acid molecule. In one embodiment, the method involves administering an agent (e.g., an agent identified by a screening assay described herein), or combination of agents that modulates (e.g., upregulates or downregulates) marker protein expression or activity. In another embodiment, the method involves administering a marker protein or nucleic acid molecule as therapy to compensate for reduced or aberrant marker protein expression or activity.

Stimulation of marker protein activity is desirable in situations in which marker protein is abnormally downregulated and/or in which increased marker protein activity is likely to have a beneficial effect. For example, stimulation of marker protein activity is desirable in situations in which a marker is downregulated and/or in which increased marker protein activity is likely to have a beneficial effect. Likewise, inhibition of marker protein activity is desirable in situations in which marker protein is abnormally upregulated and/or in which decreased marker protein activity is likely to have a beneficial effect.

3. Pharmacogenomics

The marker protein and nucleic acid molecules of the present invention, as well as agents, or modulators which have a stimulatory or inhibitory effect on marker protein activity (e.g., marker gene expression) as identified by a screening assay described herein can be administered to individuals to treat (prophylactically or therapeutically) marker-associated disorders (e.g., a Th1- or Th2-associated condition) associated with aberrant marker protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a marker molecule or marker modulator as well as tailoring the dosage and/or therapeutic regimen of treatment with a marker molecule or marker modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11) :983-985 and Linder, M. W. et aL (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of subjects taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a marker protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a marker molecule or marker modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a marker molecule or marker modulator, such as a modulator identified by one of the exemplary screening assays described herein.

This invention is further illustrated by the following examples which should not be construed as limiting. The contents of all references, patents and published patent applications cited throughout this application, as well as the Figures and the Tables are incorporated herein by reference.

### EXAMPLES

### Example 1

### Identification and Characterization of Maker cDNA

A. Isolation of Naïve CD4⁺ T Cells

Blood was obtained from the North London Blood Transfusion Service (London, UK) or from umbilical cords of neonates (Royal Free Hospital, London, UK, Chelsea and Westminster Hospital, London, UK). PBMC were isolated using Lymphoprep (Nycomed, Oslo, Norway) and washed in HBSS (PAA Laboratories, Linz, Austria) three times. CD4⁺ T cells were purified by immunomagnetic separation using M-450 CD4 Dynabeads (Dynal, Oslo, Norway). Umbilical cord blood CD4⁺ cells isolated by this method were >96% CD4⁺ and were all of naïve phenotype. CD4⁺CD45RA+T cells (e.g., naïve T cells) were purified from adult blood by incubating with monoclonal antibodies specific for UCHL 1 (anti-CD45RO-10 μg/1.0×10⁷ target cells) for 30 min. Antibody-bound cells were washed three times in HBSS supplemented with 2.5% FCS and then incubated with sheep anti-mouse IgG Dynabeads (Dynal, Oslo, Norway). CD4⁺CD45RA+ cells were negatively selected by immunomagnetic separation and were stained and analyzed by flow cytometry (FACS scan, Becton Dickinson, San Jose, Calif. USA) and were found to be greater than 95% CD45RA+.

B. Isolation of RNA and Preparation of labeled Microarray Probes

Total RNA was isolated from cells using the RNeasy mini kit (Qiagen, Hilden, Germany). To prepare cRNA for hybridization, 3-5 μg of total RNA was denatured at 70 ° C. with T7-tagged oligo-dT primer, cooled on ice, then reverse transcribed with 200 units Superscript RT II at 50 ° C. for 1 hour in 1× first-strand buffer, 10 mM DTT and 0.5 mM of each dNTP (Gibco BRL, Gaithersburg, Md.). Second strand cDNA was synthesized by adding 40 units DNA pol I, 10 units E. coli DNA ligase, 2 units RNase H, 30 μL second strand buffer, 3 μl 10 mM each dNTP, and water to 150 μL final volume and incubating at 15.8° C. for 2 hours. The resulting cDNA was extracted once with phenol/chloroform/isoamylalcohol. CDNA was separated on a Phase Lock Gel tube at maximum speed for 2 min and precipitated with sodium acetate and 100% ethanol. The resulting pellet was washed with 80% ethanol, was dried and was resuspended in diethylpyrocarbonate-treated (DEPC-treated) water.

Labeled RNA was prepared from clones containing a T7 RNA polymerase promoter site by incorporating labeled ribonucleotides in an in vitro transcription (IVT) reaction. Half of the purified cDNA was used for in vitro transcription with a T7 RNA polymerase kit, following manufacturer instructions and using an overnight 37 degree C incubation, thereby incorporating biotinylated CTP and UTP. Labeled RNA was purified using RNeasy columns (Qiagen). RNA was concentrated and the quantitated by spectrophotometry. Labeled RNA (13-15 μg) was fragmented in 40 mM Tris-acetate 8.0, 100 mM potassium acetate, 30 mM magnesium acetate for 35 min at 94 ° C. in a total volume of 40 μL.

C. Array Hybridization and Detection of Fluorescence

The labeled and fragmented RNA probes were diluted in 1× MES buffer, B10948, Bio C, B cre, 100 μg/ml herring sperm DNA, and 50 μg/ml acetylated BSA. New probes were pre-hybridized in a microfuge tube with glass beads at 45 ° C. overnight to remove debris. Oligonucleotide arrays composed of 6800 human genes (Microarray, Affymetrix, LocN, Cat. No. 51037) were pre-hybridized with 1× MES hybridization buffer at 45 ° C. for 5 min and then insoluble material was removed by centrifugation. Pre-hybridization buffer was removed from oligo array cartridges, 200 μL probe added and cartridges were hybridized for 16 hours at 45 ° C. at 60 rpm. After hybridization, probes were removed and the cartridges washed extensively with 6× SSPET using a fluidics station (Affymetrix). Following hybridization, the solutions were removed, the arrays were washed with 6×SSPE-T at 22° C. for 7 min, and then washed with 0.5× SSPE-T at 40 ° C. for 15 minutes. When biotin-labeled RNA was used, the hybridized RNA was stained with a streptavidin-phycoerythrin conjugate (Molecular Probes, Eugene, Oreg.) prior to reading. Hybridized arrays were stained with 2 μg/ml streptavidin-phycoerythrin in 6× SSPE-T at 40° C. for 5 minutes. The arrays were read using a scanning confocal microscope made for Affymetrix by Molecular Dynamics (commercially available through Affymetrix, Santa Clara, Calif.) The scanner uses an argon ion laser as the excitation source, with the emission detected by a photomultiplier tube through either a 530 nm bandpass filter (fluorescein), or a 560 nm longpass filter (phycoerythrin). Nucleic acids of either sense or antisense orientations were used in hybridization experiments. Arrays with probes for either orientation (reverse complements of each other) are made using the same set of photolithographic masks by reversing the order of the photochemical steps and incorporating the complementary nucleotide.

D. Quantitative Analysis of hybridization Patterns and Intensities

Following a quantitative scan of an array, a grid is aligned to the image using the known dimensions of the array and the comer control regions as markers. The image is reduced to a simple text file containing position and intensity information using software developed at Affymetrix (GENECHIP 3.0 software). This information is merged with another text file that contains information relating physical position on the array to probe sequence and the identity of the RNA and the specific part of the RNA for which the oligonucleotide probe is designed. The quantitative analysis of the hybridization results involves a simple form of pattern recognition based on the assumption that, in the presence of a specific RNA, the PM probes will hybridize more strongly on average than their MM partners. The number of instances in which the PM hybridization signal is larger than the MM signal is computed along with the average of the logarithm of the PM/MM ratios for each probe set. These values are used to make a decision (using a predefined decision matrix) concerning the presence or absence of an RNA. To determine the quantitative RNA abundance, the average of the differences (PM minus MM) for each probe family is calculated. The advantage of the difference method is that signals from random cross-hybridization contribute equally, on average, to the PM and MM probes, while specific hybridization contributes more to the PM probes. By averaging the pairwise differences, the real signals add constructively while the contributions from cross-hybridization tend to cancel. When assessing the differences between two different RNA samples, the hybridization signals from side-by-side experiments on identically synthesized arrays are compared directly. The magnitude of the changes in the average of the difference (PM−MM) values is interpreted by comparison with the results of spiking experiments as well as the signals observed for the internal standard bacterial and phage RNAs spiked into each sample at a known amount. Data analysis programs developed at Affymetrix, such as the GENECHIP 3.0 software, perform these operations automatically.

To generate the data in Table 1, genes were clustered hierarchically into groups on the basis of similarity of their expression profiles by the procedure of Eisen et al. ((1998) Proc. Natl. Acad. Sci. USA 95(25):14863-8). In the present example, DNA microarrays representing 6800 unique full-length gene sequences were used to analyze the pattern of gene expression in human Th1 and Th2 cell populations generated in vitro. The experiments described were designed to map gene expression during the early stages of T helper cell differentiation and compare these results to differentiated and restimulated Th1 and Th2 cell populations. Genes that were designated absent in all samples in a given experiment were eliminated from the analysis, as were -fold changes over the designated baseline of less than 2. Genes that were designated absent in all samples were eliminated. Differences in fold change between Th1 and Th2 samples were calculated by subtraction. Affymetrix software uses two different algorithms for determining the presence or absence of a gene and the increase or decrease over baseline. Therefore, in some cases a gene will be represented as an increase but is called absent. In addition, a gene may be significantly increased over the baseline but be designated to be unchanged. Therefore, genes that fit both of these criteria are those considered to be of most interest for further analysis.

Table 1 lists the genes which were found to have expression levels that were increased or decreased by at least a factor of 2 from naïve CD4⁺ T cells, grouped into categories based on the known function of the gene. The actual measurements of gene expression (in terms of -fold change) in Th1 and Th2 cells after 24 hours of Th1-inducing or Th2-inducing treatment are shown in the columns marked “Th1 24h” and “Th2 24 h”, respectively. A difference call (e.g., increased, decreased, or no change) for each gene in each of Th1 and Th2 cells was also made, and is set forth in Table 1. Table 2 includes those genes from Table 1 in which a gene was observed to have decreased expression in Th1 but unchanged expression in Th2. Table 3 includes those genes from Table 1 in which a gene was observed to have increased expression in Th1 but unchanged expression in Th2. Table 4 includes those genes from Table 1 in which a gene was observed to have unchanged expression in Th1 cells but decreased expression in Th2 cells. Table 5 includes those genes from Table 1 with unchanged expression in Th1 cells but increased expression in Th2 cells. Table 6 includes only those genes which have changed expression (either increased or decreased) in both Th1 and Th2 cells, as compared to naïve CD4⁺ T cells.

E. Time Course of Gene Expression in Naïve CD4⁺T Cells in Response to Th1-Inducing and Th2-Inducing Conditions

A preliminary experiment was designed to analyze the changes in gene expression over a 24 hour period in response to Th1-inducing or Th2-inducing conditions. Naïve CD4⁺ T cells isolated from cord blood of three different donors were cultured for the indicated times in the presence of microbeads coated with anti-CD3 and anti-CD28 and 10 ng/ml recombinant(r) human IL-2. Such conditions induce a rapid and prolonged proliferation of these cells. Cells were cultured in the presence of either 10 ng/ml rIL-12 and 200 ng/ml anti-IL-4 (Th1-inducing conditions) or 10 ng/ml rIL-4 and 2 μg/ml anti-IL-12 (Th2-inducing conditions). RNA was isolated from naïve CD4⁺ T cells and cell cultures at the indicated times and individual donors were pooled. Fluorescent RNA probes were made from these pools and used to probe the Affymetrix human 6800 DNA microarray set.

Gene expression levels were assessed using Affymetrix software. The relative abundance, designated the ‘average difference’, of a particular gene was calculated from the intensity of hybridization to a perfect matched set of oligonucleotides compared to a corresponding set of single base mismatched oligonucleotides. Genes were designated as either present or absent according to their average difference. In order to compare the relative abundance of transcripts between samples, the naïve CD4⁺ T cell RNA sample was designated as the baseline and the average difference of each gene in all other samples was compared to it. A total of 775 genes (˜12%) were designated as present in the naïve CD4⁺ T cell baseline. The numbers of genes designated present increased relative to the length of stimulation, reaching a maximum after 24 hours (Th1-inducing conditions for 24 hours, 1193; Th2-inducing conditions for 24 hours, 1261).

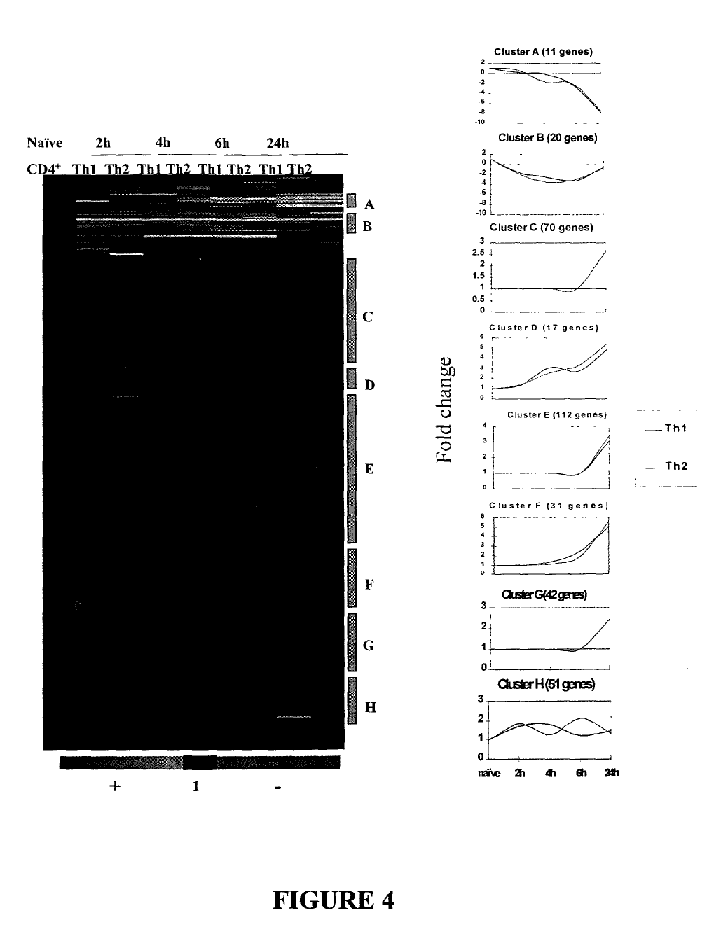

A hierarchical cluster algorithm was used to group genes or treatments with similar expression patterns (data not shown). Clustering samples by treatment group resulted in treatments being clustered according to length of stimulation rather than stimulation conditions, indicating that most genes are commonly expressed in Th1-inducing and Th2-inducing conditions in the first 24 hours of stimulation (data not shown). Therefore, treatment groups were clustered by gene expression similarities. Clusters of both immediate early and late responsive genes were identified. The expression of 99 genes was shown to increase or decrease during the first 6 hours of differentiation, indicating that they are involved in the early phase of Th1 and Th2 differentiation (FIG. 4—Clusters A, B, D and H). The majority of these genes did not differ from the naïve CD4⁺ T cell baseline by more than 2 fold. These clusters included known immediate-early genes such as JunB, pim1, c-myc and STAT5. Of these immediate-early genes, only 11 genes could be classified as Th1 specific, including hCREM (cyclic AMP-responsive element modulator), and 16 genes could be classified as Th2 specific, including the IL-4 receptor (FIG. 4—Cluster H).

A group of 112 genes which were increased in both samples after 24 hours, but were not specific to Th1 or Th2 differentiation, were likely to be involved in T effector cell differentiation (FIG. 4—Cluster E). This group included genes such as cyclin, several ATP synthase genes, several cytochrome C oxidase genes and several proteosome subunit genes. A cluster of 42 genes was shown to be differentially expressed after 24 hours in Th1-inducing conditions and included genes such as IFN-γ , STAT1 and TAP1, which is involved in antigen presentation (FIG. 4—Cluster G). A cluster of 70 genes was shown to be differentially expressed after 24 hours in Th2-inducing conditions and included genes such as GATA-3, the Th2-specific transcription factor.

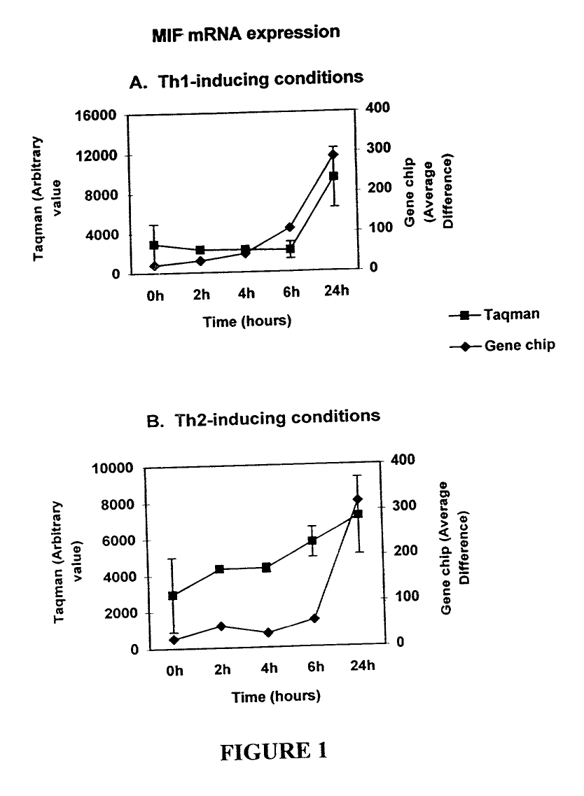

RNA pooled from three individual donors for each time point was used for the DNA array analysis in order to determine whether gene expression levels detected were representative of each sample, and to independently verify the relative gene expression levels detected on the DNA microarrays using another method of assessing gene expression. Macrophage inhibitory factor (MIF) gene expression was selected for further analysis by PCR as MIF mRNA was shown to increase in both Th1-inducing and Th-2 inducing conditions over 24 hours by DNA array analysis (FIG. 4—Cluster F). Gene expression levels for each of the three individual RNA samples making up a single time point for Th1 and Th2 cultures were measured using the Taqman 5′ nuclease fluorogenic quantitative PCR assay (FIG. 1). Expression of MIF mRNA during Th1-inducing (FIG. 1A) or Th2-inducing conditions (FIG. 1B) was shown to increase over time in both Th1 and Th2 samples as assessed by DNA microarray analysis. The relative increase in MIF mRNA detected by PCR was shown to correlate with the DNA microarray levels in Th1-inducing conditions (FIG. 1A). During Th2-inducing conditions, greater variation between the three samples was observed but relative MIF mRNA levels were consistent between PCR and DNA microarray data. Previous studies indicate that DNA microarray analysis using Affymretix DNA microarrays give both ratios and absolute mRNA levels that are comparable to traditional methods of mRNA detection. These data demonstrate that DNA microarray analysis is a reliable method of analysing gene expression levels and also that there was not significant variation in gene expression among the three donor samples.

F. Patterns of Gene Expression in Naïve CD4⁺ T Cells in Response to Th1-Inducing and Th2-Inducing Conditions

In light of the previous results, a stimulation time of 24 hours was selected for a more detailed analysis of the differences in gene expression in Th1-inducing versus Th2-inducing conditions. A second experiment was performed on naïve CD4′ T cells from a single donor. Cells were stimulated for 24 hours using anti-CD3 and anti-CD28 microbeads and 10 ng/ml of IL-2 and either 10 ng/ml IL-12 and 200 ng/ml anti-IL-4 (Th1-inducing conditions) or 10 ng/ml IL-4 and 2 μg/ml anti-IL-12 (Th2-inducing conditions).

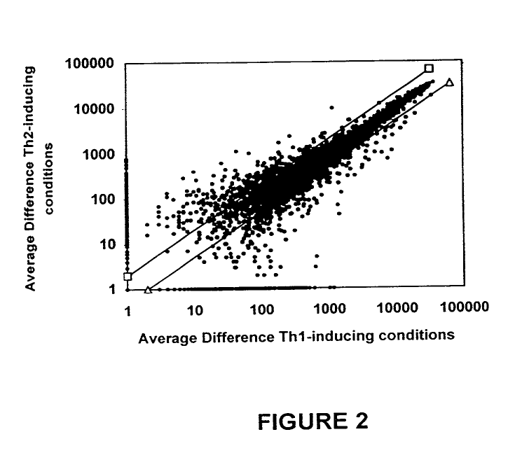

Average differences for each gene in Th1-inducing and Th2-inducing conditions were plotted against each other (FIG. 2). Genes with average differences less than or equal to 0 were defaulted to 1. Lines drawn on the graph represent greater than 2 fold increase in Th1 compared to Th2-inducing conditions (Λ) or a greater than 2 fold increase in Th2 compared to Th1-inducing conditions (□). The majority of these genes, located between the two parallel lines, were less than two fold different between Th1 and Th2-inducing conditions. Genes with average differences that were greater than two fold different between Th1 and Th2-inducing conditions fell outside the two lines and were considered to be of most interest for further study. Highly expressed genes with a large average difference, for example ribosomal protein S11 (average difference Th1-29911, Th2 -25621), were mainly housekeeping genes. Likewise, genes with lower average differences, such as IFN-γ (Th1-1455, Th2-44) and GATA-3 (Th1-309, Th2-1326), were greater than two fold different between the two samples and were likely to be important in the differentiation of Th1 and Th2 cells.

G. Analysis of Individual Gene Expression in Naïve CD4⁺ T Cells in Response to Th1-Inducing and Th2-Inducing Conditions

After 24 hours stimulation, CD4′ T cells stimulated in Th1-inducing or Th2-inducing conditions do not display cytokine secretion profiles typical of differentiated T helper cells. Previous studies have demonstrated that production of IFN-γ and IL-4 is cell cycle dependent. IFN-γ was produced after just one cell division (Day 1), whereas IL-4 production requires at least four cell divisions (Day 3). In the present example, increased expression of IFN-γ mRNA (15.7 fold increase over baseline) was detected in Th1-inducing conditions and was absent in Th2-inducing conditions (Table 1). IL-12Rβ2 mRNA expression exhibited a similar pattern of expression. This finding is in agreement with previous studies, which showed that the IL-12Rβ2 chain is up-regulated on CD4⁺ T cells in response to stimulation via the T cell receptor, is maintained in response to IL-12, but it is down regulated in response to IL-4. IL-4 mRNA could not be detected after 24 hours in Th2-inducing conditions but in contrast, IL-4 receptor mRNA was detected. IL-4 receptors are present on resting T cells and addition of exogenous IL-4 results in up-regulation of IL-4 receptor mRNA. GATA-3 mRNA could be detected in the naïve CD4⁺ T cell baseline sample, was absent in Th1-inducing conditions and was increased in Th2-inducing conditions (Table 1). GATA-3 has been reported to be present in naïve CD4⁺ T cells and is down regulated in cells differentiating along the Th1 pathway. The impact of the relative amounts of mRNA produced in particular cell types is difficult to determine. Post-translational modifications and mRNA stability will impact on the levels of particular proteins produced. Analysis of relative IFN-γ protein levels demonstrated a correlation between mRNA and protein expression (data not shown).

Many genes from different functional categories were found to be differentially expressed in either Th1-inducing or Th2-inducing conditions. Chemokines are a family of molecules that control the migration of leukocytes into tissues in response to physiological and inflammatory conditions. Th1 and Th2 cells have been shown to express overlapping and distinct sets of chemokine receptors that are now known to govern their specific migration into particular inflammatory sites. Th1 cells preferentially express CXCR3 and CCR5, while Th2 cells preferentially express CR3 and CCR4. Analysis of gene expression using DNA microarrays demonstrated that CCR7 was differentially expressed in Th2-inducing conditions after 24 hours. CCR7 has been shown to be expressed by naïve CD4⁺ T cells and is thought to be involved in directing T cells into the lymph node where they are primed by antigen. Differentiated Th1 cells in mice were shown to preferentially express CCR7 mRNA and localized to the periarteriolar lymphoid sheath. In contrast, activated Th2 cells that lacked CCR7 expression homed to the periphery of the T cell zone, which is in close proximity to the B cell zone. CCR7 surface expression was up-regulated to a higher level on a Th2 polarized cell line compared to a Th1 polarized cell line in the first 24 hours of T cell activation, followed by a decrease in CCR7 on both cell types after 24 hours. The present study demonstrated that stimulation of naïve CD4⁺ T cells in Th2-inducing conditions up-regulated CCR7 mRNA expression, at least in the first 24 hours of stimulation and together with previous studies may indicate that IL-4 is capable of rapidly and transiently up-regulating CCR7 on Th2 cells. Further analysis of chemokine receptor mRNA expression in response to IL-4 would be needed to confirm this finding. The transient expression of CCR7 mRNA demonstrates that chemokine receptor expression is very flexible and may play an important role in regulating lymphocyte traffic.

Expression of chemokine mRNA was also detected in both Th1-inducing and Th2-inducing conditions. MIP-1α and MIP-1β mRNA, which are induced by both IL-12 and IFN-γ, were differentially expressed in Th1-inducing conditions. MIP-3α, which is IFN-inducible, was also preferentially induced in response to Th1-inducing conditions. MIP-3α is a chemoattractant for lymphocytes and monocytes. It has also been shown to preferentially attract immature Langerhans dendritic cells. IFN-γ induced production of MIP-3α in Th1 cells at peripheral sites of inflammation may provide a means of recruiting immature dendritic cells to that site. In contrast, TARC was differentially expressed in Th2-inducing conditions. TARC binds to the CCR4 receptor, which is preferentially expressed on Th2 cells. The production of these chemokines by T cells may represent a means of further up-regulating chemokine receptor expression.

There is emerging evidence that epigenetic regulation of gene expression can confer inheritance of a lymphocyte phenotype. Epigenetic constraints on the bulk structure of chromatin can limit the accessibility of genes and restrict their transcription. Relieving epigenetic repression by hypermethylation of histones and demethylation of DNA can allow genes to become transcriptionally active. Recent studies have demonstrated that chromatin remodelling of cytokine gene loci is functionally associated with T helper cell differentiation. Differentiation of naïve T cells into Th1 or Th2 cells was associated with differential chromatin accessibility of the IFN-γ and IL-4 loci, respectively. Treatment of cells with inhibitors of histone deacetylase and cytosine methylase was shown to increase IFN-γ and IL-4 production (not shown). Chromatin remodelling of the IL-4 locus occurs within 48 hours at a time when IL-4 transcripts are barely detectable.

In this example, SMARCB1 and SMARCA2, two actin-dependent regulators of chromatin that are involved in reversing chromatin-dependent inhibition of transcription, were differentially expressed in Th2-inducing and Th1-inducing conditions, respectively. These molecules have been shown to alter chromatin structure and increase DNA accessibility in an ATP-dependent manner (reviewed in Workman and Kingston, 1998). Specific molecules involved in chromatin remodelling of IFN-γ and IL-4 loci have not so far been identified and it is possible that SMARCB1 and SMARCA2 are specifically involved in this process.

H. Taqman Polymerase Chain Reaction

To ensure that the data obtained from the GeneChip analysis (described above) was reflective of the actual level of gene expression in the cell samples, the expression of a selected gene, MIP, was also measured by polymerase chain reaction, and the results compared to the expression levels observed by GeneChip analysis. Total RNA was treated with 10 units of RQ1 DNase I (Promega, Madison, Wis., USA) for 30 min at 37 ° C. Samples were extracted with phenol/chloroform, and RNA was precipitated with 0.3 M sodium acetate and 2 volumes of 100% ethanol. RNA was resuspended in DEPC-treated water, and the RNA concentration determined by measuring the optical absorbance at 260 nm. Then rTth DNA polymerase was used to reverse transcribe and amplify 25 ng of total RNA in a single tube assay using the Perkin-Elmer TaqMan EZ RT-PCR kit (Perkin-Elmer Applied Biosystems, Foster City, Calif., USA) with gene-specific sense and anti-sense primers and a probe fluorescently labeled at the 5′ end with 6-carboxyl-fluorescein (6-FAM) (Kruse (1997) J Immunol. Methods 210(2):195-203; Heid et al. (1996) Genome Res 6(10):986-94). Primers and fluorescently labeled probes were generated using Primer Express software (Perkin-Elmer), and were synthesized by Perkin-Elmer. To avoid amplification of contaminating genomic DNA, primer pairs crossing intron/exon boundaries were selected. Duplicate samples were reverse transcribed for 30 min at 60 ° C. and then subjected to 40 rounds of amplification for 15 sec at 95 ° C. and 1 min at 60 ° C. using the ABI Prism 7700 sequence detection system as described by the manufacturer (Perkin-Elmer) (Kruse et al. (1997) supra). Sequence-specific amplification was detected as an increased fluorescence signal of 6-FAM during the amplification cycle. Quantitation of gene-specific message levels was based on a comparison of the fluorescence intensity in the unknown mRNA sample to the fluorescence intensity from the standard curve of known mRNA levels. Amplification of the gene for human acidic ribosomal protein (HARP) was performed on all samples tested to control for variations in RNA amounts. MIF mRNA was normalized to this control mRNA. The results are shown in FIG. 1, and demonstrate that the data obtained from the GeneChip analysis accurately reflects the expression level of the genes in the cellular samples.

I. Discussion

1. Role of Interferon-Inducible Genes in Th1 Differentiation

A distinct program of IFN-inducible gene expression in response to Th1-inducing conditions after 24 hours was identified. The interferon family has been shown to induce the expression of some 200 genes in many different cell types. This pattern of gene expression indicates that one or more of the interferon molecules may play an important role in the early differentiation of Th1 cells. Expression of IL- 12Rβ2 chain is critical for Th1 development and has been shown to be induced by IL-12, and extinguished in the presence of IL-4. IL-12 induced expression of IL-12Rβ2 and activation of STAT4 has been shown to be IFN-γ dependent. IFN-γ mRNA expression in T cells has previously been shown to be induced by IL-12 within 6 hours. IFN-γ has also been shown to induce high levels of STAT1 mRNA. In this example, STAT1 mRNA expression was preferentially up-regulated in Th1-inducing conditions compared to Th2-inducing conditions after 24 hours. STATI is activated by phosphorylation in response to IFN-γ and occurs within 15 to 30 minutes after IFN-γ treatment. STATI is then translocated to the nucleus where it acts as a transcriptional activator of a number of genes. The DNA binding activity of STAT1 disappears within hours. Induction of STAT1 transcription either by IFN-γ or IL-12 may serve as an additional point of control for this potent transcriptional activator.

STAT1 levels have been shown to be negatively regulated by ubiquitin-dependent proteolysis. Three genes involved in protein degradation, UBE1L, UBE2D1 and a proteosome subunit, were shown to be differentially expressed in Th1-inducing conditions. These proteins may be involved in regulating active STAT1 levels by protein degradation. Therefore, STATI activity could be regulated by IFN-γ or IL-12 or both at a transcriptional level by enhancing mRNA accumulation and also post-translationally by promoting proteolysis and degradation.

Most of the signals shown to be important in Th1 and Th2 differentiation are applicable to both mouse and human cells, with one notable exception. Both IFN-α and IFN-β, the type I IFNs, can directly induce human Th1 development. In contrast, type I IFN's can not induce mouse Th1 differentiation. The demonstration that IFN-α/β could induce STAT-4 activation in human but not mouse T cells provided an explanation for this species-specific difference.

A family of transcription factors, termed the interferon regulatory factors (IRFs) were differentially expressed in Th1-inducing conditions in the present study. IRF-1, ISGF3G and ICSBP were all preferentially expressed in Th1-inducing conditions. IRF-1 and ISGF3G are positive regulators of IFN-stimulated genes, whereas ICSBP acts as a transacting negative regulator by interacting with these two proteins. IRF-1 gene expression has been shown to be up-regulated by IL-12 via STAT4 activation in T cells and was not mediated indirectly by IFN-α. IFN-γ inducible gene (L07633) and IP30 were preferentially induced in Th1-inducing conditions, and have been shown to be induced by IFN-γ in another study using Affymetrix DNA micorarrays to compare gene expression in response to IFN-α, -β and -γ in a fibrosarcoma cell line. Der et al. demonstrated that each IFN regulates a distinct set of IFN-inducible genes. In particular, MX1, G1P2 were induced by IFN-α and -β only, whereas PPP3CA was induced by IFN-α and down-regulated by IFN-β. In the present study, the induction of MX1 and GIP2, together with the down-regulation of PPP3CA in Th1-inducing conditions, indicates that type I IFN's may be acting at the early stages of Th1 differentiation in human T cells.

A set of IFN-inducible genes involved in antigen presentation were also differentially expressed in Th1-inducing conditions. The role of IFN-γ in inducing MHC molecule expression is well-documented. Two MHC class one genes were shown to be differentially expressed in Th1-inducing conditions. MHC class I genes are constitutively expressed in T cells but can still be significantly up-regulated by IFN-γ. TAP1 and TAP2 are encoded in the MHC region of the genome, and are closely linked to LMP2 and LMP7, two proteosomal polymorphic genes. All four genes are induced by IFN-γ. Many genes within this region are known to be regulated by IFN-γ via interaction with a short, bi-directional sequence motif termed the interferon stimulated response element (ISRE). The differential expression of genes in the MHC region in Th1-inducing conditions may reflect co-regulation of distinct members of this gene class as a result of their use of common promoters.

### Example 2

### Analysis of Gene Expression in Restimulated Fully-differentiated TH1 and TH2 Cells

A. Stimulation of Differentiation

Gene expression in Th1 and Th2 cells under conditions which would normally stimulate these cells was examined. Purified CD4+CD45RA+ T cells were cultured in 6-well flat-bottom tissue culture plates (Falcon, Becton Dickinson Labware, N.J., USA), seeded at a density of 2.0×10⁶ cells/ml in RPMI 1640 (PAA Laboratories, Linz, Austria) supplemented with 10% human serum (Biowhittaker, Walkersville, Md., USA), 100 μg/ml penicillin and streptomycin (OAA Laboratories, Linz, Austria) and 2 μM L-glutamine (PAA Laboratories, Linz, Austria). Cells were stimulated with M-450 tosylactivated Dynabeads (Dynal, Oslo Norway) coated with anti-CD3 and anti-CD28 (Levine (1995) Int. Immunol. 7: 891-904) using one bead/cell, and 10 ng/ml rIL-2, 10 ng/ml rIL-12 and 200 ng/ml anti-IL-4 (for stimulation of Th1 differentiation), or 10 ng/ml IL-4 and 2 μg/ml anti-IL-12 (for stimulation of Th2 differentiation). After 7 days of culture, anti-CD3/anti-CD28 coated beads were removed by incubating cells in 10 μL DETACHaBEAD per 1.0×10⁷ cells for 2 hours and then washed three times in HBSS supplemented with 2.5% FCS (Sigma, St. Louis, Mo., USA). Cells were subsequently restimulated with 50 ng/ml 4-phorbol-12-myristate 13-acetate (PMA) and 250 ng/ml calcium ionophore ionomycin for four hours. Cells were collected by centrifugation and washed once with phosphate buffered saline prior to isolation of RNA and marker analysis as above.

A number of genes were identified that had significantly (e.g., 2-fold or greater) increased or decreased expression relative to naïve CD4⁺ T cells. These genes are set forth in Tables 7 and 13. Table 8 includes those genes from Table 7 which were increased in expression in Th1 cells and unchanged in expression in Th2 cells. Table 9 includes those genes from Table 7 which were decreased in expression in Th1 cells and increased in expression in Th2 cells. Table 10 includes those genes from Table 7 which were increased in Th2 cells but which were unchanged in expression in Th1 cells. Table 11 includes those genes from Table 7 which were changed in expression (e.g., increased or decreased) in both Th1 and Th2 cells relative to naïve T cell controls.

B. Confirmation of Differentiation

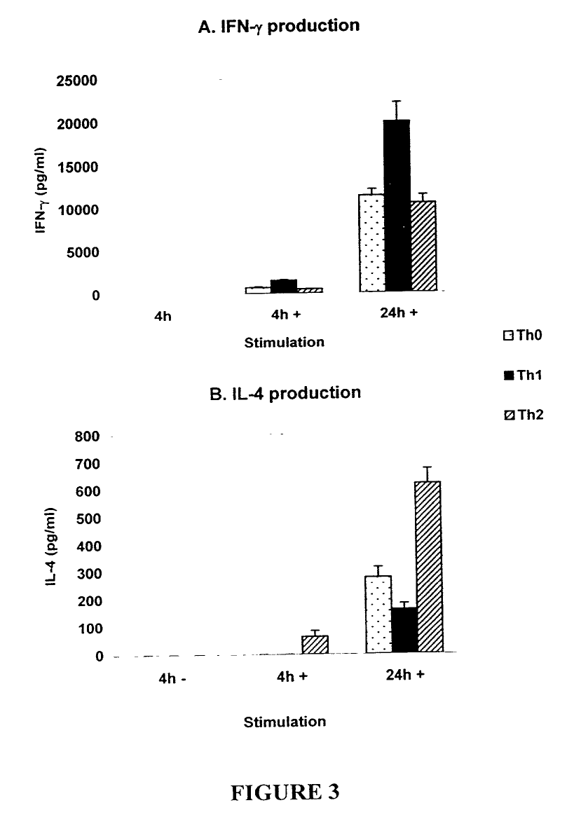

To confirm that the treatments applied to naïve T cells to induce differentiation into either Th1 or Th2 cells resulted in the appropriate cell differentiation being induced, an experiment in which cytokines expected to be produced by each cell type were measured was performed. The isolated CD4+ naïve T cells were induced to differentiate into either Th1 or Th2 cells by incubation with appropriate cytokines. Naïve T cells were treated for 7 days with 10 ng/ml rIL-2, 10 ng/ml rIL-12 and 200 ng/ml anti-IL-4 (for stimulation of Th1 differentiation), or with 10 ng/ml IL-4 and 2 μg/ml anti-IL-12 (for stimulation of Th2 differentiation). Subsequent to this incubation, cells were stimulated for 4 hours with PMA or ionomycin, as described above. Th1 cells typically secrete IFN-γ and Th2 cells typically secrete IL-4; therefore, IFN-γ (FIG. 3A) and IL-4 (FIG. 3B) production in the culture supernatant were assayed by ELISA. The results, shown in FIG. 3, represent the mean and standard deviation of triplicate samples and are representative of several experiments. As is clear from the graphs, the Th1 cells secreted significantly more IFN-γ than either the naïve T cells (Tₒ) or Th2 cells, and Th2 cells secreted significantly more IL-4 than either naïve T cells (Tₒ) or Th1 cells.

C. Discussion

1. Patterns of gene Expression in Differentiated Thi and Th2 Cell Populations

This example demonstrates how the DNA microarray technology can be use to analyze gene expression in differentiating cells at particular time points. The patterns of gene expression in differentiated Th1 and Th2 cell populations were compared to gene expression in the first 24 hours of differentiation.

In order to control for genes induced in response to stimulation with anti-CD3, anti-CD28 and rIL-2, naïve T cells were stimulated in the absence of T helper-inducing cytokines and anti-cytokine antibodies. Naïve CD4⁺ T cells were stimulated for 7 days with anti-CD3 and anti-CD28 coated microbeads and 10 ng/ml IL-2 (Th0 conditions) and either 10 ng/ml rhu IL-12 and 200 ng/ml anti-IL-4 (Th1 conditions) or 10 ng/ml rhu IL-4 and 2 μg/ml anti-IL-12 (Th2 conditions). After 7 days, microbeads were removed and cells washed thoroughly in media for two hours to remove exogenous cytokines. Each population was then either stimulated for 4 hours with media alone or for 4 and 24 hours with 50 ng/ml PMA and 250 ng/ml ionomycin. Differentiation of cell populations into the Th0 , Th1 or Th2 phenotype was confirmed by ELISA assay of 4 and 24 hour culture supernatants (FIG. 3). None of the cell populations expressed IFN-γ or IL-4 in the absence of mitogenic stimulation. Cells cultured in the presence of anti-CD3 and anti-CD28 coated microbeads and rIL-2 expressed IFN-γ after 4 hours restimulation and both IFN-γ and IL-4 after 24 hours restimulation. This cell population expressed less IFN-γ than Th1 cells and less IL-4 than Th2 cells and was therefore designated Th0 cells. Cells cultured in Th1 conditions produced more IFN-γ after 4 and 24 hours stimulation than either the Th0 or Th2 cell populations, no IL-4 after 4 hours stimulation and a lower level of IL-4 than either Th0 or Th2 cells after 24 hours stimulation. Cells cultured in Th2 conditions produced more IL-4 than Th0 and Th1 conditions after both 4 and 24 hours restimulation.

RNA prepared from cell cultures restimulated for 4 hours in the presence of mitogens was used to generate fluorescent probes that were hybridized to Affymetrix 6800 microarrays overnight. Fluorescence patterns were detected using a laser scanner and the results expressed as fold change over the naïve CD4⁺ T cell baseline. In order to eliminate genes that were induced in response to the culture conditions, both Th1 and Th2 cell fold changes were subtracted from Th0 fold changes. Only those genes that were greater than two fold different from Th0 cells in either Th1 and Th2 cells were considered (Tables 7-11).

Analysis of cytokine expression at the protein level demonstrated that cell populations had differentiated to a Th1 and Th2 cell phenotype. Restimulation of differentiated Th1 and Th2 cells for 4 hours resulted in differential expression of IFN-γ and IL-2 mRNA in Th1 cells and IL-4 and IL-13 mRNA in Th2 cells (Table 7). Mitogenic restimulation is clearly important for distinguishing between in vitro differentiated Th1 and Th2 cells on the basis of cytokine profile. Analysis of cytokine mRNA secretion profiles at the single cell level have shown that individual clones from a mixed population of cells vary widely in the combinations and amounts of cytokines expressed. The analysis presented here indicates that there are relatively few differences in Th1 and Th2 cells at the molecular level. Since many Th1 and Th2 specific genes are induced by IFN-γ or IL-4, this analysis will not necessarily identify these genes because Th1 and Th2 gene expression has been subtracted from a background of Th0 cells, which express both of these cytokines. Analysis of cloned T helper cell lines may reveal more diversity in gene expression between the two cell types. More importantly, this experiment is likely to reflect the situation in vivo where Th1 and Th2 cell development occurs gradually over time and T helper cells found in inflammatory sites represent a gradation of differentiation with respect to cytokine secretion profiles.

2. Analysis of Individual Gene Expression in Differential Th1 and Th2 Cell Populations

Analysis of gene expression in Th1 and Th2-inducing conditions demonstrated that a number of chemokines are preferentially up-regulated in these cell types. Of those differentially expressed after 24 hours, only MIP-3α was also found to be preferentially expressed in differentiated Th1 cells. IFN-γ induced production of MIP-3α in Th1 cells at peripheral sites of inflammation may provide a means of recruiting immature dendritic cells to that site.

ANX2, or lipocortin II, is a calcium-regulated integral membrane binding protein. Lipocortin II promotes cellular proliferation and has been shown to increase osteoclast formation and bone resorption. Lipocortin II/Annexin 2, also known as phosphoprotein 36, has been shown to induce Th2 immune responses when injected into mice. In the present study, lipocortin II is preferentially expressed in Th2 cells. Several studies have shown that IL-4 can promote osteoclast development of monocytes and monocyte cell lines but another study indicates that IL-4 inhibits bone resorption in an ex vivo model. It is possible that lipocortin II production is induced by IL-4 in T cells and osteoclast formation and bone resorption could be promoted by IL-4-induced lipocortin II.

A number of genes not previously known to be preferentially expressed by Th1 or Th2 cells were identified in this analysis. HSPA1L, known as heat shock protein (hsp) 70, was up-regulated to a higher degree in Th0 and Th2 cells compared to Th1 cells. Hsps are ubiquitously expressed molecular chaperones that are involved in many cellular functions. The immunogenicity of heat shock proteins is well-documented but the role of hsp produced by lymphocytes has not been addressed. Production of specific heat shock proteins by memory T cells in response to particular cytokines could serve a means of amplifying the immune response at particular sites of inflammation. The recent finding that hsp70 can act as a cytokine and induce the production of pro-inflammatory cytokines in monocytes indicates that hsp70 may be involved in eliciting immune responses. A G-protein molecule known as G-α 16 was up-regulated in Th2 cells, compared to Th0 and Th1 cells. G-α 16 protein is specifically expressed in hematopoietic cells and may possibly be involved in IL-2 signalling. Increased expression of G-α 16 protein in Th2 cells may mean that this signalling molecule is involved in Th2-specific cytokine gene expression.

TABLE 1
Changes in gene expression after 24 hours In Th1-inducing or Th2-inducing conditions.

| Accession | | | Th1 | Th1 | Th2 | Th2 |
|---|---|---|---|---|---|---|
| Number | Gene | Description | 24 h | I/D | 24 h | I/D |
| | | Antigen Presentation | | | | |
| L06175 | RH17599 | MHC class I region ORF | >4.2^{a} | 1^{b} | *^{a} | NC^{b} |
| M31525 | HLA-DNA | MHC class II lymphocyte antigen | >1 | NC | >3 3 | I |
| 129376 | MHC/FRAG | MHC class I mRNA fragment | >2 1 | NC | * | NC |
| M74447 | TAP2 | TAP2 Atp-binding Cassette Protein | >11 2 | I | * | NC |
| X00274 | HLAORA | MHC class II alpha heavy chain | * | NC | −3 1 | D |
| X56841 | HLAE | HLA-E | 1 | NC | −3 | D |
| HG4724-HT5166 | TAP1 | Atp-Binding Cassette Protein | 23 | I | 5 8 | I |
| HG4724-HT5166 | TAP1 | Atp-Binding Cassette Protein | 28 | I | 4 4 | I |
| HG3576-HT3779 | W528 | Major Histocompatibility Complex | −2 3 | NC | * | D |
| | | Cell Death | | | | |
| D0001 | APT1 | APO-1 antigen | >4 6 | I | >1 | NC |
| M63379 | CLO | Clusterin, APOJ | * | NC | >4.2 | I |
| U28014 | CASP4 | Caspase 4 | 3 9 | I | 1 | NC |
| | | Cell Division | | | | |
| D63743 | CENPE | mitotic centromere-associated kinesin | >3.4 | I | * | NC |
| HG4120-HT4392 | CDC2LI | CDC-2 like protein kinase | 2 9 | NC | * | D |
| | | Cell Structure/organelles | | | | |
| D84454 | UGALT | UDP-galactose translocator | I | NC | −3 | NC |
| X98534 | VASP | vasodilator-stimulated phosphoprotein | >5 4 | I | * | NC |
| L03785 | MYL5 | regulatory myosin light chain | >1 | NC | >3.6 | I |
| D03851 | CAPZA | Capping protein alpha | 3.1 | I | 1 | NC |
| X81438 | AMPH | Amphiphysin | * | NC | >5.3 | I |
| X64838 | RSN | H.sapiens mRNA for restin | >4.7 | I | 1 | NC |
| | | Cell Surface Receptors/Proteins | | | | |
| L48211 | AGTR2 | Angiotensin receptor II | >2.7 | NC | * | NC |
| D28137 | BST2 | BST-2 bone marrow stromal cell antigen 2 | >4.4 | I | >1 | NC |
| X69920 | CALCR | Calcitonin receptor | >1 | NC | >6.3 | I |
| Y00636 | D058 | D058-LFA-3 | >3.2 | I | >1 | NC |
| M62403 | IGFBP4 | Insulin-like growth factor binding protein 4 | >4.8 | NC | * | NC |
| X13916 | LRP1 | LOL-receptor related protein | >5.5 | I | * | NC |
| 120852 | SLC20A2 | Leukemia virus receptor 2, P transporter | >2.1 | NC | * | NC |
| X96719 | AlCL | Activation-induced lectin | 2 | I | −2.6 | 0 |
| M16336 | D02 | T cell surface antigen CD2 | 3.9 | I | 1 | I |
| M13560 | D074 | MHC class II invariant polypeptide | 3.5 | I | 1 | NC |
| U76764 | CD97 | Leokocyte activation antigen CD97 | 1 | NC | −3.8 | D |
| X81479 | EMR1 | EMR1 hormone receptor | 3.4 | I | * | D |
| M65085 | FSHR | Follicle stimulating hormone receptor | * | NC | 3 | NC |
| M58285 | HEM1 | membrane-assoc. haemopoetic protein | 15.7 | I | 3.9 | I |
| X53586 | ITGA6 | integrin alpha 6 | 1 | NC | * | D |
| 106797 | L5 | LS orphan G protein-coupled receptor | −3.1 | D | 1 | NC |
| D72661 | NINJ1 | Ninjurn 1 nerve injury-induced protein | 1 | NC | * | D |
| M54927 | PLP | Myelin proteolipid protein | * | D | * | NC |
| 128175 | PTGER2 | Prostaglandin E2 receptor EP2 subtype | 1 | NC | * | NC |
| 104953 | X11 | Amyloid beta (M) precursor protein-binding | * | NC | 1 | NC |
| | | Chemokines/Receptors | | | | |
| M28130 | IL8 | Interleukin 8 | 1 | D | −6.8 | D |
| D43767 | TARO | thymus and activation regulated chemokine | * | NC | >3.7 | MI |
| D64197 | SCYA20 | MIP-3(x chemokine (IFN-inducible) | >3.7 | I | * | NC |
| 108177 | CMKBR7 | CCR7 chemokine receptor | * | D | 3.4 | I |
| M23178 | SCYA3 | MIP-1α chemokine | I | NC | −3.1 | D |
| J04130 | SCYA4 | MIP-1β chemokine | 2 | NC | * | MD |
| | | Chromatin and Nuclear Structure | | | | |
| 004847 | SMARCB1 | SWI/SNF related, matrix associated, actin | >1 | NC | >3.1 | NC |
| | | dependent regulator of chromatin | | | | |
| X72889 | SMARCA2 | SWI/SNF related, matrix associated, actin | 3.1 | I | 1 | NC |
| | | dependent regulator of chromatin. subfamily | | | | |
| | | b, member 1 | | | | |
| | | Cytokines/Receptors | | | | |
| X04500 | IL1B | Interleukin-1β | −2.4 | D | * | D |
| X52425 | IL4R | Interleukin 4 receptor | 1 | I | 3.7 | I |
| U64198 | IL12RB2 | lnterleukin-12 receptor β2 chain | >13.1 | I | * | NC |
| V00536 | IFNG | Interferon-gamma | 15.6 | I | −1.6 | NC |
| | | Inteferon-inducible | | | | |
| L07633 | IFNGI-5111 | interferon-gamma 1GUP 1-5111 protein | 15.7 | I | * | NC |
| U22897 | NDP52 | Nuclear domain 10 protein (ndp52) | 3.2 | I | 1 | NC |
| M55642 | GBPI | Guanylate binding protein isoform I | >18.9 | I | >2.7 | I |
| M55543 | GBP2 | Guanylate binding protein isoform II | >43 | I | >13.0 | I |
| M91196 | ICSBP1 | IFN consensus sequence binding protein | 24.7 | I | 4.9 | I |
| U72882 | IF135 | interferon-induced leucine zipper protein | >34 | I | >8.2 | I |
| X02530 | INP10 | IFN-y inducible early response gene | >3.5 | NC | * | NC |
| J03909 | IP30 | IFN-y inducible protein (lP-30) | >3.3 | I | * | NC |
| M33882 | MX1 | p78 GTP binding protein | >7 6 | I | * | NC |
| M13755 | G1P2 | interferon-induced I7Kd/15Kd protein | 46 | I | 1 | NC |
| X57351 | INP18D | 1-8D, interferon-inducible gene family | 3 7 | I | I | NC |
| L05072 | IRF1 | interferon regulatory factor 1 | 7 | I | 1 | NC |
| M87503 | ISGF3G | IFN-responsive transcription factor | 3 8 | I | 1 | NC |
| | | Metabolic Enzymes | | | | |
| HG1828-HT1857 | NXGLD | Nexin, Glia-Derived | >3 1 | I | >1 | I |
| K03192 | CYP2A6 | Cytochrome P450, hA | * | NC | >2 2 | NC |
| M90516 | GFPT | Glutamine fructose-6-phosphate | >1 | NC | >3 6 | I |
| | | amidotransferase (GFAT) | | | | |
| D16480 | HADHA | mitochobdrial enoyl-CoA hydratase | >22 7 | I | >6 5 | I |
| U18932 | HSST | Heparan sulfate-N-deacetylase/N- | >2 6 | NC | * | NC |
| | | sulfotransferase | | | | |
| M93405 | MMSDH | Methylmalonate semialdehyde | * | NC | >3 2 | NC |
| | | dehydrogenase | | | | |
| 120971 | PDE4B | Phosphodiesterase 4B | >2 4 | I | * | NC |
| ACOO2115 | COX6B | COX6B, Cytochrome C oxidase | * | NC | 3 2 | NC |
| 125798 | HMGCS1 | 3-hydroxy-3-methylglutaryl coenzyme A | 3 1 | I | 1 | I |
| | | synthase | | | | |
| AF0050 | PARG | Poly(ADP-ribose) glycohydrolase (hPARG) | 3 7 | I | 1 | NC |
| S41458 | PDE6B | Phosphodiesterase 6B | 3 8 | Ml | 1 | NC |
| L35594 | PDNP2 | Phosphodiesterase I | 1 | NC | * | D |
| D49817 | PFKFB1 | Fructose 6-phosphate,2-kinaseffructose | * | MD | * | D |
| X90858 | UP | Uridine phosphorylase | 1 | NC | * | D |
| | | Protein Degradation | | | | |
| HG1649-HT1652 | ELAl | Elastase 1 | >9 7 | I | >33.3 | I |
| L13852 | UBElL | Ubiquitin-activating enzyme El related | >3 | I | >1 | NC |
| | | protein | | | | |
| HG3344-HT3521 | UBE2D1 | Ubiquitin-Conjugating Enzyme Ubch5 | >3.7 | I | >1 | I |
| X71874 | PSMB10 | Proteasome (prosome. macropain) subunit | 55.8 | I | 14.8 | I |
| | | RNA Processing | | | | |
| 137127 | POLR2B | RHA polymerase II | 23.1 | I | 4.8 | I |
| J03798 | SNRPN | autoantigen small nudear ribonucleoprotein | * | D | 1 | NC |
| | | Signal Transduction | | | | |
| U78095 | PLBK | Placental bikunin (Serine protease inhibitor) | >1 | NC | >12.7 | I |
| 000017 | ANX2 | Lipocortin Il (phospholipase A2 inhibitor) | >4.5 | NC | >1 | NC |
| 010495 | PRKCO | Protein kinase C delta-type | >3.5 | NC | >1 | NC |
| HG3187-HT336 | PTPN1 | non-receptor protein tyrosine phosphatase | >7 1 | NC | * | NC |
| K03218 | SRC | c-src-1 proto-oncogene | >4.9 | MI | * | I |
| 138529 | STAT1 | (done NSA) protein p84 | >93.9 | I | >13.1 | 1 |
| 1570426 | A28RGS14P | p53 target gene | 3.2 | I | 1 | Ml |
| 1548807 | DUSP4 | MAP kinase phosphatase (MKP-2) | 1 | NC | 3.3 | I |
| 1578575 | PIP5K1A | 88 kDa type I phosphatidylinositol-4- | 3.3 | MI | 1 | NC |
| | | phosphate 5-kinase alpha | | | | |
| L14778 | PPP3CA | Calmodulin-dependent protein phosphatase | * | D | 1 | NC |
| | | catalytic subunit (PPP3CA) | | | | |
| X89416 | PPP5C | protein phosphatase 5 | 3.7 | 1 | 29.8 | I |
| S59049 | RGS1 | B cell activation gene regulator of G-protein | 1 | NC | −8.1 | D |
| | | signalling | | | | |
| | | Transcription Factors | | | | |
| HG4036-HT4306 | RB1 | Retinoblastoma 1 | 3.7 | MI | 1 | NC |
| HG4036-HT4306 | RB1 | Retinablastoma 1 | 4.5 | NC | 1 | NC |
| D89377 | MSX2 | Drosophila homeo box homolog 2 | * | NC | >3.8 | I |
| U26173 | NFIL3 | NF-IL3A - basic ZIP protein | >1 | I | >4.6 | I |
| U44848 | NRF1 | Nuclear respiratoiy factor I (NRF1) | >2.5 | NC | * | NC |
| U49082 | PBX2 | pre-B-cell leukemia transcription factor 2 | >3.5 | I | * | NC |
| U00115 | BCL6 | bcl6-zinc finger protein | 1 | NC | * | D |
| U23736 | GATA3 | GATA-3 | * | NC | 5.5 | NC |
| U20734 | JUNB | JunB | −3.6 | 0 | −64.4 | 0 |
| U19067 | RELA | NF-kB p65 - RelA | * | D | * | NC |
| X98253 | ZNF183 | Zinc finger protein RING finger | 1 | NC | * | D |
| | | Micellaneous | | | | |
| X15673 | PTR2 | pTR2 mRNA for repetitive sequence. | * | NC | * | D |
| M11119 | PU | endogenous retrovirus envelope region | * | NC | 1 | NC |
| V00594 | MT2A | metallothionein from cadmium-treated cells | 8.3 | I | 2.3 | NC |
| HG627-HT5098 | RHV3 | Rhesus (Rh) Blood Group System Antigen | * | NC | >4.4 | MI |
| X07315 | PP15 | PP15 (nuclear import protein) | >1 | NC | >3.3 | Ml |
| U97018 | EMAPL | echinoderm microtubule-assoc. protein | * | NC | >3.4 | I |
| | | homolog | | | | |
| L15409 | VI-IL | von Hippel-Lindau syndrome gene | * | NC | >3 1 | I |
| X99140 | H85 | Keratin | * | NC | >5.5 | Ml |
| U90552 | BTN5 | Butyrophilin (BTF5) | >3.7 | I | >1 | I |
| U01212 | OMP | Olfactory marker protein (OMP) | * | NC | >3.4 | NC |
| Z50194 | PQRICH | PQ-nch protein | * | NC | >4 8 | I |
| U35048 | TSC22 | TGF-g-stimulated proteinTSC-22 | >3 | I | >1 | NC |
| X67698 | TISSP | Epididymal secretory protein - tissue | 4 3 | I | 1 | I |
| | | specific | | | | |
| | | Unknown Function | | | | |
| U90547 | RORET | Ro/SSA nbonucleoprotein homolog | >1 | NC | >3 1 | I |
| 082070 | AC1 | Clone expressed in neuroblastoma cell line | * | NC | >3 6 | NC |
| U66052 | E_W2_6 | X chromosome unknown clone | >1 | NC | >4.5 | MI |
| S83364 | RAB5IP | putative RabS-interacting protein | 12.3 | I | 3.1 | NC |
| D87017 | IGL2 | (lambda) DNA for immurloglobin light chain | * | NC | >7.9 | NC |
| D50918 | K128 | KIAA0128 | >7.3 | I | >2.1 | I |
| D31886 | K66 | KIAA0066 | >3.6 | NC | * | NC |
| D87449 | K260 | KIAA0260 | 2.5 | NC | * | NC |
| AB0023 | K317 | KIAAO317 | 4.5 | I | 1 | NC |
| D25304 | K6 | KIAA0006 | 3.2 | I | 1 | I |
| Fold changes^{a} and difference calls^{b} (I - increase, D - decrease, NC - no change) were calculated for Th1-inducing and Th2-inducing conditions based on comparison to the naive CD4⁺ T cell baseline. | | | | | | |
| Only fold changes that were <−2 or >2 between the two samples were considered. Genes designated as absent in the baseline sample by analysis of hybridisation to positive and negative oligonucleotides are represented by * instead of fold change because accurate fold | | | | | | |
| #changes can not be calculated when comparing to absent genes. A > indicates that the fold change is based on an absent gene in the baseline sample and therefore is likely to be higher. Where two copies of a gene were detected, only one is shown. | | | | | | |

TABLE 2
Changes in gene expression after 24 hours In Th1-inducing or Th2-inducing conditions:
Genes decreased in expression in Th1 cells and unchanged in expression in Th2 cells

| Accession | | | Th1 | Th1 | Th2 | Th2 |
|---|---|---|---|---|---|---|
| Number | Gene | Description | 24 h | I/D | 24 h | I/D |
| L06797 | L5 | L5 orphan G protetn-coupled receptor | −3 1 | D | 1 | NC |
| M54927 | PLP | Myelin proteolipid protein | * | D | * | NC |
| | | RNA Processing | | | | |
| J03798 | SNRPN | autoantigen small nuclear ribonucleoprotein | * | D | 1 | NC |
| | | Signal Transduction | | | | |
| L14778 | PPP3CA | Calmodulin-dependent protein phosphatase | * | D | 1 | NC |
| | | catalytic subunit (PPP3CA) | | | | |
| | | Transcription Factors | | | | |
| L19067 | RELA | NF-kB p65 - RelA | * | D | * | NC |
| Fold changes^{a} and difference calls^{b} (I - increase, D - decrease, NC - no change, MI - moderate increase, MD - Moderate decrease) were calculated for Th1-inducing and Th2-inducing conditions based on comparison to the naive CD4 between the two samples were considered. Genes designated as absent in the baseline sample by analysis of hybridisation to positive and negative oligonucleotides are represented by instead of fold change because accurate fold | | | | | | |
| #changes can not be calculated when comparing to absent genes A > indicates that the fold change is based on an absent gene in the baseline sample and therefore is likely to be higher Where two copies of a gene were detected, only one is shown. | | | | | | |

TABLE 3
Changes In gene expression after 24 hours In Th1-Inducing or Th2-lnducing conditions:
Genes Increased In expression In Th1 cells and unchanged In expression In Th2 cells

| Accession | | | Th1 | Th1 | Th2 | Th2 |
|---|---|---|---|---|---|---|
| Number | Gene | Description | 24 h | I/D | 24 h | I/D |
| | | Antigen Presentation | | | | |
| L06175 | RH17599 | MHC class I region ORF | >4 2^{a} | I^{b} | *^{a} | NC^{b} |
| M74447 | TAP2 | TAP2 Atp-binding Cassette Protein | >11 2 | I | * | NC |
| | | Cell Death | | | | |
| D0001 | APT1 | APO-1 antigen | >4 6 | 1 | >1 | NC |
| U28014 | CASP4 | Caspase 4 | 3 9 | I | 1 | NC |
| | | Cell Division | | | | |
| U63743 | CENPE | mitotic centromere-associated kinesin | >3 4 | 1 | * | NC |
| | | Cell Structure/organelles | | | | |
| X98534 | VASP | vasodilator-stimulated phosphoprotein | >5.4 | I | * | NC |
| U03851 | CAPZA | Capping protein alpha | 3 1 | I | 1 | NC |
| X64838 | RSN | H. sapiens mRNA for restin | >4 7 | I | 1 | NC |
| | | Cell Surface Receptors/Proteins | | | | |
| 028137 | BST2 | BST-2 bone marrow stromal cell antigen 2 | >4 4 | I | >1 | NC |
| Y00636 | CD58 | CD58 - LFA-3 | >3 2 | I | >1 | NC |
| X13916 | LRP1 | LOL-receptor related protein | >5 5 | I | * | NC |
| M13560 | D074 | MHC class II invariant polypeptide | 3 5 | I | 1 | NC |
| | | Chemokines/Receptors | | | | |
| U64197 | SCYA20 | MIP-3ct chemokine (IFN-inducible) | >3 7 | I | * | NC |
| | | Chromatin and Nuclear Structure | | | | |
| X72889 | SMARCA2 | SWI/SNF related, matrix associated, actin | 3 1 | I | 1 | NC |
| | | dependent regulator of chromatin, subfamily | | | | |
| | | b, member 1 | | | | |
| | | Cytokines/Receptors | | | | |
| U64198 | IL12RB2 | Interleukin-12 receptor β2 chain | >13 1 | I | * | NC |
| V00536 | IFNG | Interferon-gamma | 15 6 | 1 | −1.6 | NC |
| | | Interferon-inducible | | | | |
| 107633 | IFNG 1-5111 | Interferon-gamma IGUP I-5111 protein | 15 7 | I | * | NC |
| U22897 | NDP52 | Nuclear domain 10 protein (ndp52) | 3.2 | I | I | NC |
| J03909 | 1P30 | IFN-γ inducible protein (IP-30) | >3.3 | I | * | NC |
| M33882 | MX1 | p78 GTP binding protein | >7.6 | I | * | NC |
| M13755 | G1P2 | interferon-induced 17Kd/15Kd protein | 4.6 | I | 1 | NC |
| X57351 | INP18D | 1-8D, interferon-induable gene family | 3.7 | I | I | NC |
| 105072 | IRF1 | interferon regulatory factor 1 | 7 | I | 1 | NC |
| M87503 | ISGF3G | IFN-responswve transcription factor | 3.8 | I | 1 | NC |
| | | Metabolic Enzymes | | | | |
| 120971 | PDE4B | Phosphodiesterase 4B | >2.4 | I | * | NC |
| AF0050 | PARG | Poly(ADP-ribose) glycohydrolase (hPARG) | 3.7 | I | 1 | NC |
| L13852 | UBEl L | Ubiquitin-activating enzyme E1 related | >3 | I | >1 | NC |
| | | protein | | | | |
| | | Signal Transduction | | | | |
| U70426 | A28RGS14P | p53 target gene | 3.2 | I | 1 | Ml |
| | | Transcription Factors | | | | |
| U49082 | PBX2 | pre-B-cell leukemia transcription factor 2 | >3.5 | 1 | * | NC |
| | | Miscellaneous | | | | |
| V00594 | MT2A | metallothionein from cadmium-treated cells | 8.3 | I | 2.3 | NC |
| U35048 | TSC22 | TGF-β-stimulated proteinTSC-22 | >3 | I | >1 | NC |
| | | Unknown Function | | | | |
| S83364 | RAB5IP | putative Rab5-interacting protein | 12.3 | 1 | 3.1 | NC |
| A80023 | K317 | KIAA0317 | 4.5 | I | I | NC |
| Fold changes^{a} and difference calls^{b} (I - increase, D - decrease, NC - no change, MI - moderate increase, MD - Moderate decrease) were calculated for Th1-inducing and Th2-inducing conditions based on comparison to the naive CD4 between the two samples were considered. Genes designated as absent in the baseline sample by analysis of hybridisation to positive and negative oligonucleotides are represented by instead of fold change because accurate fold | | | | | | |
| #changes can not be calculated when comparing to absent genes A > indicates that the fold change is based on an absent gene in the baseline sample and therefore is likely to be higher Where two copies of a gene were detected, only one is shown. | | | | | | |

TABLE 4
Changes in gene expression after 24 hours in Th1-inducing or Th2-inducing conditions:
Genes unchanged in expression in TH2 cells and decreased in expression in TH2 cells

| Accession | | | Th1 | Th1 | Th2 | Th2 |
|---|---|---|---|---|---|---|
| Number | Gene | Description | 24 h | I/D | 24 h | I/D |
| | | Antigen Presentation | | | | |
| X00274 | HLADRA | MHC class II alpha heavy chain | * | NC | −3 1 | D |
| X56841 | HLAE | HLA-E | 1 | NC | −3 | 0 |
| HG3576-HT3779 | W52B | Major Histocompatibiltty Complex | −2 3 | NC | * | D |
| | | Cell Division | | | | |
| HG4120-HT4392 | CDC2L1 | CDC-2 like protein kinase | 2 9 | NC | * | D |
| | | Cell Surface Receptors/Proteins | | | | |
| U76764 | 0097 | Leokocyte activation antigen CD97 | 1 | NC | 3 8 | D |
| X53586 | ITGA6 | integrin alpha 6 | 1 | NC | * | D |
| U72661 | NINJ1 | Ninjurin 1 nerve injury-induced protein | 1 | NC | * | D |
| | | Chemokines/Receptors | | | | |
| M23178 | SCYA3 | MIP-1α chemokine | 1 | NC | −3 1 | D |
| | | Metabolic Enzymes | | | | |
| L35594 | PDNP2 | Phosphodiesterase I | 1 | NC | * | D |
| D49817 | PFKFB1 | Fructose 6-phosphate,2-kinase/fructose | * | MD | * | D |
| X90858 | UP | Uridine phosphorylase | 1 | NC | * | D |
| | | Signal Transduction | | | | |
| S59049 | RGS1 | B cell activation gene regulator of G-protein | 1 | NC | −8 1 | D |
| | | signalling | | | | |
| | | Transcription Factors | | | | |
| U00115 | BCL6 | bcl6 - zinc finger protein | 1 | NC | * | D |
| X98253 | ZNFI83 | Zinc finger protein RING finger | 1 | NC | * | D |
| | | Miscellaneous | | | | |
| X15673 | PTR2 | pTR2 mRNA for repetitive sequence | * | NC | * | D |
| Fold changes^{a} and difference calls^{b} (I - increase, D - decrease, NC - no change, MI - moderate increase, MD - Moderate decrease) were calculated for Th1-inducing and Th2-inducing conditions based on comparison to the naive CD4 between the two samples were considered. Genes designated as absent in the baseline sample by analysis of hybridisation to positive and negative oligonucleotides are represented by instead of fold change because accurate fold | | | | | | |
| #changes can not be calculated when comparing to absent genes A > indicates that the fold change is based on an absent gene in the baseline sample and therefore is likely to be higher Where two copies of a gene were detected, only one is shown. | | | | | | |

TABLE 5
Changes in gene expression after 24 hours in Th1-inducing or Th2-inducing conditions:
Genes unchanged in expression in Th1 cells and increased in expression in Th2 cells

| Accession | | | Th1 | Th1 | Th2 | Th2 |
|---|---|---|---|---|---|---|
| Number | Gene | Description | 24 h | I/D | 24 h | I/D |
| | | Antigen Presentation | | | | |
| M31525 | HLA-DNA | MHC class II lymphocyte antigen | >1 | NC | >3 3 | I |
| | | Cell Death | | | | |
| M63379 | CLU | Clusterin, APOJ | * | NC | >4 2 | I |
| | | Cell Structure/organelles | | | | |
| L03785 | MYLS | regulatory myosin light chain | >1 | NC | >3 6 | I |
| X81438 | AMPH | Amphiphysin | * | NC | >5 3 | I |
| | | Cell Surface Receptors/Proteins | | | | |
| X69920 | CALCR | Calcitonin receptor | >1 | NC | >6.3 | I |
| | | Metabolic Enzymes | | | | |
| M90516 | GFPT | Glutamine:fructose-6-phosphate | >1 | NC | >3 6 | I |
| | | amidotransferase (GFAT) | | | | |
| | | Signal Transduction | | | | |
| U78095 | PLBK | Placental bikunin (Serine protease inhibitor) | >1 | NC | >12 7 | I |
| K03218 | SRC | c-src-1 proto-oncogene | >4.9 | Ml | * | I |
| L36529 | STAT1 | (clone N5-4) protein p84 | >93.9 | I | >13.1 | I |
| U48807 | DUSP4 | MAP kinase phosphatase (MKP-2) | * | 1 | NC | 3.3 |
| | | Transcription Factors | | | | |
| D89377 | MSX2 | Drosophila homeo box homolog 2 | * | NC | >3.8 | I |
| | | Miscellaneous | | | | |
| U97018 | EMAPL | echinoderm microtubule-assoc. protein | * | NC | >3.4 | I |
| | | homolog | | | | |
| L15409 | VHL | von Hippel-Lindau syndrome gene | * | NC | >3.1 | 1 |
| Z50194 | PQRICH | PQ-rich protein | * | NC | >4.8 | 1 |
| | | Unknown Function | | | | |
| U90547 | RORET | Ro/SSA ribonucleoprotein homolog | >1 | NC | >3.1 | I |
| Fold changes^{a} and difference calls^{b} (I - increase, D - decrease, NC - no change, MI - moderate increase, MD - Moderate decrease) were calculated for Th1-inducing and Th2-inducing conditions based on comparison to the naive CD4 between the two samples were considered. Genes designated as absent in the baseline sample by analysis of hybridisation to positive and negative oligonucleotides are represented by instead of fold change because accurate fold | | | | | | |
| #changes can not be calculated when comparing to absent genes A > indicates that the fold change is based on an absent gene in the baseline sample and therefore is likely to be higher Where two copies of a gene were detected, only one is shown. | | | | | | |

TABLE 6
Changes In gene expression after 24 hours in Th1-inducing or Th2-inducing conditions:
Genes changed in expression In both Th1 and Th2 cells

| Accession | | | Th1 | Th1 | Th2 | Th2 |
|---|---|---|---|---|---|---|
| Number | Gene | Description | 24 h | I/D | 24 h | I/D |
| | | Antigen Presentation | | | | |
| HG4724-HT5166 | TAP1 | Atp-Binding Cassette Protein | 23 | I | 5 8 | I |
| HG4724-HT5166 | TAP1 | Atp-Binding Cassette Protein | 28 | I | 4 4 | I |
| | | Cell Surface Receptors/Proteins | | | | |
| X96719 | A1CL | Activation-induced lectin | 2 | I | −2 6 | D |
| M16336 | CD2 | T cell surface antigen CD2 | 3 9 | I | 1 | I |
| M58285 | HEM1 | membrane-assoc haemopoetic protein | 15 7 | I | 3 9 | I |
| | | Chemokines/Receptors | | | | |
| M28130 | 1L8 | interleukin 8 | 1 | D | −68 | D |
| L08177 | CMKBR7 | CCR7 chemokine receptor | * | D | 3 4 | 1 |
| | | Cytokines/Receptors | | | | |
| X04500 | IL1B | Interleukin-1β | −2 4 | D | * | D |
| X52425 | IL4R | Interleukin 4 receptor | 1 | I | 3 7 | I |
| | | Interferon-Inducible | | | | |
| M55542 | GBP1 | Guanylate binding protein isoform I | >18 9 | I | >27 I | |
| M55543 | GBP2 | Guanylate binding protein isoform II | >43 | I | >13 0 | I |
| M91196 | ICSBP1 | IFN consensus sequence binding protein | 24.7 | I | 4 9 | I |
| U72882 | IFI35 | Interferon-induced leucine zipper protein | >34 | I | >8 2 | I |
| | | Metabolic Enzymes | | | | |
| HG1828-HT1857 | NXGLD | Nexin, Glia-Derived | >3 1 | I | >1 | I |
| D16480 | HADHA | mitochobdrial enoyl-COA hydratase | >22.7 | I | >6 5 | I |
| L25798 | HMGCS1 | 3-hydroxy-3-methylglutaryl coenzyme A | 3 1 | I | 1 | I |
| | | synthase | | | | |
| | | Protein Degradation | | | | |
| HG1649-HT1652 | ELA1 | Elastase 1 | >9.7 | I | >33 3 | I |
| HG3344-HT3521 | UBE2D1 | Ubiquitin-Conjugating Enzyme Ubch5 | >3.7 | I | >1 | I |
| X71874 | PSMB10 | Proteasonie (prosome, macropain) subunit | 55.8 | I | 14 8 | I |
| | | RHA Processing | | | | |
| L37127 | POLR2B | RNA polymerase II | 23 | 1 | I | 4.8 |
| | | Signal Transduction | | | | |
| L36529 | STAT1 | (done N5-4) protein p84 | >93.9 | I | >13.1 | 1 |
| X89416 | PPP5C | protein phosphatase 5 | 3.7 | I | 29.8 | I |
| | | Transcription Factors | | | | |
| U26173 | NFIL3 | NF-IL3A - basic ZIP protein | >1 | I | >4.6 | I |
| U20734 | JUNB | JunB | −3.6 | D | −64.4 | D |
| | | Miscellaneous | | | | |
| U90552 | BTN5 | Butyrophilin (BTF5) | >3.7 | I | >1 | I |
| X67698 | TISSP | Epididymal secretory protein - tissue | 4.3 | I | 1 | I |
| | | specific | | | | |
| | | Unknown Function | | | | |
| D50918 | K128 | KIAA0128 | >7.3 | I | >2.1 | I |
| D25304 | K6 | KIAA0006 | 3.2 | I | 1 | I |
| Fold changes^{a} and difference calls^{b} (I - increase, D - decrease, NC - no change, MI - moderate increase, MD - Moderate decrease) were calculated for Th1-inducing and Th2-inducing conditions based on comparison to the naive CD4 between the two samples were considered. Genes designated as absent in the baseline sample by analysis of hybridisation to positive and negative oligonucleotides are represented by instead of fold change because accurate fold | | | | | | |
| #changes can not be calculated when comparing to absent genes A > indicates that the fold change is based on an absent gene in the baseline sample and therefore is likely to be higher Where two copies of a gene were detected, only one is shown. | | | | | | |

TABLE 7
ComparIson of gene expression between cells differentiated in Th1-inducing and
Th2-inducing conditions and then restimutated for 4 hours.

| Accession | | | Th0 | Th1 | Th2 |
|---|---|---|---|---|---|
| number | Gene | Description | 4 h restim | 4 h restim | 4 h restim |
| J00314 | E_TUBB | beta-tubulin | >3.0^{a} | >27 | >5.9 |
| M94345 | CAPG | macrophage capping protein | >6.5 | >17 | >87 |
| AF008445 | PLSCR1 | phospholipid scramblase | 2 2 | 1 3 | 5 7 |
| M11717 | HSPA1L | heat shocl4 protein (hsp 70) | 13 3 | 3 7 | 10 5 |
| X01630 | ASS | argininosuccinate synthetase | >3 8 | >1 4 | >3 9 |
| M63904 | GNA16 | G-alpha 16 protein | >1 | >4 0 | >10 8 |
| D38583 | S100A11 | calgizzarin | 65 | 32 | 77 |
| S77835 | IL2 | interleukin 2 | 24 2 | 47 2 | 21 7 |
| V00536 | IFNG | interferon-gamma | 23 5 | 42 9 | 21 4 |
| X00371 | MB | myoglobin gene | 5 5 | 2 3 | 5 2 |
| U64197 | SCYA20 | MIP-3α | 1.9 | 4 1 | 17 |
| M13982 | IL4 | interleukin 4 (IL-A) | 3 4 | 3 3 | 5 6 |
| U31120 | IL13 | interleukin-13 (IL-13) precursor | 4 9 | 3 4 | 8 6 |
| D49396 | APT1 | Apol | 2 4 | 2 2 | 5 3 |
| Naive CD4⁺ T cells were stimulated for 7 days an Th0. Th1 and Th2-inducing conditions and then restimulated for 4 hours with PMA and janomycan Fold changes were based on comparison to the naive CD4⁺ T cell baseline All fold changes above 2 were designated increases by Affymetrix software Only genes that were greater than two fold different from Th0-inducing conditions in | | | | | |
| #either Th-1 or Th2-inducing conditions are displayed Fold changes preceded by > indicates that the fold change is based on an absent gene in the baseline sample and therefore can not accurately be calculated | | | | | |

TABLE 8
Comparison of gene expression between cells
differentiated in Th1-inducing and Th2-inducing
conditions and then restimulated for 4 hours by
PMA/ionomycin: Genes which are increased in
expression in Th1 cells and unchanged in expression in Th2 cells

| Accession | | | Th0 | Th1 | Th2 |
|---|---|---|---|---|---|
| number | Gene | Description | 4 h restim | 4 h restim | 4 h restim |
| 577835 | IL2 | interleukin 2 | 24.2 | 47.2 | 21.7 |
| V00536 | IFNG | interferon- | 23.5 | 42.9 | 21.4 |
| | | gamma | | | |
| U64197 | SCYA20 | MIP-3α | 1.9 | 4.1 | 1.7 |
| Naive CD4⁺ T cells were stimulated for 7 days an Th0. Th1 and Th2-inducing conditions and then restimulated for 4 hours with PMA and janomycan Fold changes were based on comparison to the naive CD4⁺ T cell baseline All fold changes above 2 were designated increases by Affymetrix software Only genes that were greater than two fold different from Th0-inducing conditions in | | | | | |
| #either Th-1 or Th2-inducing conditions are displayed Fold changes preceded by > indicates that the fold change is based on an absent gene in the baseline sample and therefore can not accurately be calculated | | | | | |

TABLE 9
Comparison of gene expression between cells differentiated in Th1-inducing and
Th2-inducing conditions and then restimulated for 4 hours by PMA/ionomycin:
Genes that are Decreased in expression in Th1 cells and unchanged in
expression in Th2 cells

| Accession | | | Th0 | Th1 | Th2 |
|---|---|---|---|---|---|
| number | Gene | Description | 4 h restim | 4 h restim | 4 h restim |
| M11717 | HSPA1L | heat shock protein (hsp 70) | 13.3 | 3.7 | 10.5 |
| X01630 | ASS | arginanosuccinate synthetase | >3.8 | >1.4 | >3.9 |
| 038583 | S100A11 | calgizzarin | 6.5 | 3.2 | 7.7 |
| X00371 | MB | myoglobin gene | 5.5 | 2.3 | 5.2 |
| Naive CD4⁺ T cells were stimulated for 7 days an Th0. Th1 and Th2-inducing conditions and then restimulated for 4 hours with PMA and janomycan Fold changes were based on comparison to the naive CD4⁺ T cell baseline All fold changes above 2 were designated increases by Affymetrix software Only genes that were greater than two fold different from Th0-inducing conditions in | | | | | |
| #either Th-1 or Th2-inducing conditions are displayed Fold changes preceded by > indicates that the fold change is based on an absent gene in the baseline sample and therefore can not accurately be calculated | | | | | |

TABLE 10
Comparison of gene expression between cells differentiated In Th1-inducing and
Th2-inducing conditions and then restimulated far 4 hours by PMA/ionomycin:
Genes that are unchanged in expression In Th1 cells and
Increased in expression in Th2 cells

| Accession | | | Th0 | Th1 | Th2 |
|---|---|---|---|---|---|
| number | Gene | Description | 4 h restim | 4 h restim | 4 h restim |
| J00314 | E_TUBB | beta-tubulin | >3.0^{a} | >2.7 | >5.9 |
| AF008445 | PLSCR1 | phospholipid scramblase | 2.2 | 1 3 | 5.7 |
| M13982 | IL4 | interleukin 4 (IL-4) | 3 4 | 3 3 | 5 6 |
| U31120 | IL13 | interleukin-13 (IL-13) precursor | 4 9 | 3 4 | 8 6 |
| D49396 | APT1 | Apol | 2 4 | 2 2 | 5 3 |
| Naive CD4⁺ T cells were stimulated for 7 days an Th0. Th1 and Th2-inducing conditions and then restimulated for 4 hours with PMA and janomycan Fold changes were based on comparison to the naive CD4⁺ T cell baseline All fold changes above 2 were designated increases by Affymetrix software Only genes that were greater than two fold different from Th0-inducing conditions in | | | | | |
| #either Th-1 or Th2-inducing conditions are displayed Fold changes preceded by > indicates that the fold change is based on an absent gene in the baseline sample and therefore can not accurately be calculated | | | | | |

TABLE 11
Comparison of gene expression between cells differentiated in Th1-inducing and
Th2-lnducing conditions and then restimulated for 4 hours by PMA/ionomycin:
Genes which are changed In expression in both Th1 and Th2 cells

| Accession | | | Th0 | Th1 | Th2 |
|---|---|---|---|---|---|
| number | Gene | Description | 4 h restim | 4 h restim | 4 h restim |
| M94345 | CAPG | macrophage capping protein | >6.5 | >1.7 | >87 |
| M63904 | GNA16 | G-alpha 16 protein | >1 | >4 0 | >10 8 |
| Naive CD4⁺ T cells were stimulated for 7 days an Th0. Th1 and Th2-inducing conditions and then restimulated for 4 hours with PMA and janomycan Fold changes were based on comparison to the naive CD4⁺ T cell baseline All fold changes above 2 were designated increases by Affymetrix software Only genes that were greater than two fold different from Th0-inducing conditions in | | | | | |
| #either Th-1 or Th2-inducing conditions are displayed Fold changes preceded by > indicates that the fold change is based on an absent gene in the baseline sample and therefore can not accurately be calculated | | | | | |

TABLE 12
Genes known in the art to be altered
in gene expression in stimulated Th1 or Th2 cells

| Gene | Description | Th1 or Th2 |
|---|---|---|
| ERM | Transcription Factor ERM | Th1 |
| P38 | Proto-oncogene C-CRK | Th1 |
| JNK2 | Protein kinase JNK2 | Th1 |
| Stat 3 | Signal transducer and activator of | Th1 |
| | transcription-3 | |
| Stat 4 | Signal transducer and activator of | Th1 |
| | transcription-4 | |
| IFN-γ | γ-interferon | Th1 |
| IL-12Rβ 2 | Interleukin-12 receptor β 2 | Th1 |
| T-bet | T-box transcription factor | Th1 |
| CXCR3 | C-X-C chemokine receptor type 3 | Th1 |
| CCR7 | C-C chemokine receptor type 7 | Th1 |
| CCR5 | C-C chemokine receptor type 5 | Th1 |
| Stat 6 | Signal transducer and activator of | Th2 |
| | transcription-6 | |
| IL-4 | Interleukin-4 | Th2 |
| IL-5 | Interleukin-5 | Th2 |
| IL-13 | Interleukin-13 | Th2 |
| c-maf | Transcription factor C-MAF (proto- | Th2 |
| | oncogene) | |
| GATA-3 | Transcription factor GATA-3 | Th2 |
| CCR3 | C-C chemokine receptor type 3 | Th2 |
| CCR4 | C-C chemokine receptor type 4 | Th2 |

TABLE 13
Genes specific for either differentiated Th1 or Th2 cell populations. Numbers represent
fold change over the naive CD4⁺ T cell baseline.

| | Genbank | | | | | | |
|---|---|---|---|---|---|---|---|
| Encoded | Accession | | | Th1 | Th2 | Th1 PMA/ | Th2 PMA/ |
| protein | Number | Description | Cluster | unstim | unstim | ionomycin | ionomycin |
| LIFR | U78628 | leukemia inhibitory factor | A | 1 | 1 | 43.5 | 1 |
| | | receptor | | | | | |
| GABPA | U13044 | nuclear respiratory factor- | A | 1 | 1 | 40.3 | 1 |
| | | 2 subunit alpha | | | | | |
| INP10 | X02530 | γIFN inducible early | A | 1 | 1 | 36.9 | 1 |
| | | response gene | | | | | |
| IFNG | L07633 | interferon-gamma IEF | A | 2.8 | 1 | 33.4 | 1 |
| K166 | D79988 | KIAA0166 “gene,” | A | 1 | 2.1 | 7 | 1 |
| CYP11B1 | M32879 | steroid 11-beta- | A | 1 | 2.9 | 4.4 | 1 |
| | | hydroxylase | | | | | |
| UGCG | D50840 | ceramide | B | 0.434783 | 1 | 1 | 3.7 |
| | | “glucosyltransferase,” | | | | | |
| K5 | D13630 | KIAA0005 “gene,” | B | 1 | 1 | 1 | 2.9 |
| RTVP1 | X91911 | RTVP-1 protein | B | 1 | 1 | 1 | 3 |
| P542 | L38696 | autoantigen p542 | B | 1 | 1 | 1 | 3 |
| GOLLIMB | HG3115- | Golli-Mbp | B | 1 | 1 | 1 | 3 |
| P | HT3291 | | | | | | |
| BRCA2 | U50523 | BRCA2 “region,” mRNA | B | 1 | 1 | 1 | 3 |
| WDR2 | U57057 | WD protein IR10 | B | 1 | 1 | 1 | 3.1 |
| ARC20 | AF006087 | Arp2/3 protein complex | B | 1 | 1 | 1 | 3.3 |
| | | subunit | | | | | |
| LCP2 | U20158 | 76 kDa tyrosine | B | 1 | 1 | 1 | 3.9 |
| | | phosphoprotein SLP-76 | | | | | |
| ILA | U03397 | receptor protein 4-1BB | B | 1 | 1 | 1 | 4.2 |
| IL16 | HG270- | Lymphocyte | B | 1 | 1 | 1 | 4.3 |
| | HT270 | Chemoattractant Factor | | | | | |
| POU2F2 | M36542 | lymphoid-specific | B | 1 | 1 | 1 | 4.5 |
| | | transcription factor | | | | | |
| MB | X00371 | myoglobin gene | B | 1 | 1 | 1 | 5.2 |
| TNFR1 | M58286 | tumor necrosis factor | B | 1 | 1 | 1 | 2 |
| | | receptor | | | | | |
| UBE2L1 | S81003 | ubiquitin conjugating | B | 1 | 1 | 1 | 2 |
| | | enzyme “[human,” | | | | | |
| | | odontogenic | | | | | |
| | | “keratocysts,” mRNA | | | | | |
| K127 | D50917 | KIAA0127 | B | 1 | 1 | 1 | 2.7 |
| ALAS1 | Y00451 | 5-aminolevulinate | B | 1 | 1 | 1 | 2.8 |
| | | synthase | | | | | |
| HMGIY | L17131 | Human high mobility | B | 1 | 1 | 1 | 2.8 |
| | | group protein (HMG-I(Y)) | | | | | |
| EBVIP | U19261 | Epstein-Barr virus- | B | 1 | 1 | 1 | 2.9 |
| | | induced protein | | | | | |
| CD38 | D84276 | CD38 | F | 5 | 1 | 2.1 | 1 |
| CALR | M84739 | autoantigen calreticulin | F | 4.8 | 1 | 2.3 | 1 |
| CL1042 | X70649 | H. sapiens cl. 1042 mRNA | F | 3.2 | 1 | 2 | 1 |
| | | of DEAD box protein | | | | | |
| | | family | | | | | |
| POLR2C | J05448 | Human RNA polymerase | F | 3.7 | 1 | 2.2 | 1 |
| | | subunit hRPB “33,” | | | | | |
| AHNAKR | HG4321- | Ahnak-Related Sequence | F | 3.5 | 1 | 2.3 | 1 |
| | HT4591 | | | | | | |
| PLCG2 | D42108 | phospholipase | F | 2.1 | 1 | 2.6 | 1 |
| DG | HG1872- | Major Histocompatibility | F | 2.1 | 1 | 2.9 | |
| | HT1907 | ”Complex,” Dg | | | | | |
| CD70 | L08096 | HG903-HT903 | F | 2.8 | 1 | 3.2 | 1 |
| MTHFD | J04031 | Human | F | 3.3 | 1 | 3.9 | 1 |
| | | methylenetetrahydrofolate | | | | | |
| | | dehydrogenase- | | | | | |
| | | methenyltetrahydrofolate | | | | | |
| | | cyclohydrolase- | | | | | |
| | | formyltetrahydrofolate | | | | | |
| | | synthetase | | | | | |
| CEBPG | U20240 | C/EBP gamma | F | 2.4 | 1 | 2.6 | 1 |
| RRM1 | X59543 | M1 subunit of | F | 2.8 | 1 | 21 | 1 |
| | | ribonucleotide reductase | | | | | |
| IFI35 | U72882_at | interferon-induced leucine | F | 3 | 1 | 2.2 | 1 |
| | | zipper protein (IFP35) | | | | | |
| POLD1 | U21090_at | DNA polymerase delta | F | 2.6 | 1 | 2.1 | 1 |
| | | small subunit | | | | | |
| GLCLR | L35546_at | gamma-glutamylcysteine | F | 3.5 | 1 | 2.7 | 1 |
| | | synthetase light subunit | | | | | |
| TRANSFR | #N/A | #N/A | F | 3.3 | 1 | 2.5 | 1 |
| M | | | | | | | |
| LMNA | HG2028- | “Laminin,” A Polypeptide | F | 4 | 1 | 2.9 | 1 |
| | HT2082_at | | | | | | |
| KCNO1 | U90065_at | potassium channel | F | 2.6 | 1 | 2.4 | 1 |
| | | KCNO1 | | | | | |
| GSS | U34683_at | glutathione synthetase | F | 2.9 | 1 | 2.7 | 1 |
| IL12RB2 | U64198_at | 11-12 receptor beta2 | F | 2.9 | 1 | 2.7 | 1 |
| CYP1B1 | U03688_at | dioxin-inducible | F | 2.9 | 1 | 2.8 | 1 |
| | | cytochrome P450 | | | | | |
| CTPS | X52142_at | CTP synthetase | F | 3.2 | 1 | 2.8 | 1 |
| BLM | U39817_at | Bloom's syndrome protein | F | 3.3 | 1 | 2.8 | 1 |
| HRAS | J00277_at | c-Ha-ras1 “proto- | F | 3 | 1 | 2.5 | 1 |
| | | oncogene,” | | | | | |
| RRM2 | X59618_at | RR2 mRNA for small | F | 3.2 | 1 | 2.6 | 1 |
| | | subunit ribonucleotide | | | | | |
| | reductase | | | | | | |
| GATA3 | U23736 | | H | 1 | 1 | 0.37037 | 2.5 |
| K106 | D14662 | KIAA0106 | H | 1 | 1 | 0.212766 | 2 |
| TACT | M88282 | Human tactile protein | H | 1 | 1 | 0.25 | 2.3 |

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of assessing whether Th1 or Th2 cells are present in a subject, the method comprising comparing:
a) the level of expression of a marker in a sample from a subject, wherein the marker is selected from the group consisting of the markers listed in Tables 2-5 and 8-10, and
b) the normal level of expression of the marker in a control sample,
wherein a significant difference between the level of expression of the marker in the sample from the subject and the normal level is an indication that Th1 or Th2 cells are present in the subject.

2. The method of claim 1, wherein the marker corresponds to a transcribed polynucleotide or portion thereof, wherein the polynucleotide comprises the marker.

3. The method of claim 1, wherein the sample comprises cells obtained from the subject.

4. The method of claim 3, wherein the cells are collected from lymph.

5. The method of claim 3, wherein the cells are collected from blood tissue.

6. The method of claim 1, wherein the level of expression of the marker in the sample differs from the level of expression of the marker in naïve T cells by a factor of at least about 2.

7. The method of claim 1, wherein the level of expression of the marker in the sample differs from the level of expression of the marker in naïve T cells by a factor of at least about 5.

8. The method of claim 1, wherein the marker is not significantly expressed in tissue lacking Th1 or Th2 cells.

9. The method of claim 1, wherein the level of expression of the marker in the sample is assessed by detecting the presence in the sample of a protein corresponding to the marker.

10. The method of claim 9, wherein the presence of the protein is detected using a reagent which specifically binds with the protein.

11. The method of claim 10, wherein the reagent is selected from the group consisting of an antibody, an antibody derivative, and an antibody fragment.

12. The method of claim 1, wherein the level of expression of the marker in the sample is assessed by detecting the presence in the sample of a transcribed polynucleotide or portion thereof, wherein the transcribed polynucleotide comprises the marker.

13. The method of claim 12, wherein the transcribed polynucleotide is an mRNA.

14. The method of claim 12, wherein the transcribed polynucleotide is a cDNA.

15. The method of claim 12, wherein the step of detecting further comprises amplifying the transcribed polynucleotide.

16. The method of claim 1, wherein the level of expression of the marker in the sample is assessed by detecting the presence in the sample of a transcribed polynucleotide which anneals with the marker or anneals with a portion of a polynucleotide, wherein the polynucleotide comprises the marker, under stringent hybridization conditions.

17. The method of claim 1, comprising comparing:
a) the level of expression in the sample of each of a plurality of markers independently selected from the markers listed in Tables 2-5 and 8-10, and
b) the normal level of expression of each of the plurality of markers in samples of the same type which do not contain Th1 or Th2 cells,
wherein the level of expression of more than one of the markers is significantly altered, relative to the corresponding normal levels of expression of the markers, is an indication that Th1 or Th2 cells are present in the sample.

18. The method of claim 17, wherein the plurality comprises two or more of the markers.

19. The method of claim 17, wherein the plurality comprises at least five of the markers.

20. A method for monitoring the differentiation of naïve T cells into Th1 or Th2 cells in a subject, the method comprising:
a) detecting in a subject sample at a first point in time, the expression of a marker, wherein the marker is selected from the group consisting of the markers listed in Tables 2-5 and 8-10 and combinations thereof;
b) repeating step a) at a subsequent point in time; and
c) comparing the level of expression detected in steps a) and b), and therefrom monitoring the differentiation of naïve T cells into Th1 or Th2 cells in the subject.

21. A method for monitoring the growth and development of Th1 or Th2 cells in a subject, the method comprising:
a) detecting in a subject sample at a first point in time, the expression of a marker, wherein the marker is selected from the group consisting of the markers listed in Tables 2-5 and 8-10 and combinations thereof, wherein said group of markers does not include IFNG, SCYA20, or APT1;
b) repeating step a) at a subsequent point in time; and
c) comparing the level of expression detected in steps a) and b), and therefrom monitoring the growth and development of Th1 or Th2 cells in the subject.

22. The method of any of claims 20 or 21, wherein marker corresponds to a transcribed polynucleotide or portion thereof, wherein the polynucleotide comprises the marker.

23. The method of any of claims 20 or 21, wherein the sample comprises cells obtained from the subject.

24. The method of claim 23, wherein the cells are collected from lymph.

25. The method of claim 23, wherein the cells are collected from blood tissue.

26. A method of assessing the efficacy of a test compound or therapy for modulating differentiation of Th1 or Th2 cells in a subject, the method comprising comparing:
a) expression of a marker in a first sample obtained from the subject and exposed to or maintained in the presence of the test compound or therapy, wherein the marker is selected from the group consisting of the markers listed in Tables 2-5 and 8-10, and
b) expression of the marker in a second sample obtained from the subject, wherein the second sample is not exposed to the test compound or therapy,
wherein a significantly lower level of expression of the marker in the first sample, relative to the second sample, is an indication that the test compound or therapy is efficacious for inhibiting differentiation of Th1 or Th2 cells in the subject.

27. A method of assessing the efficacy of a test compound or therapy for modulating differentiation of Th1 or Th2 cells in a subject, the method comprising comparing:
a) expression of a marker in the first sample obtained from the subject prior to providing at least a portion of the test compound or therapy to the subject, wherein the marker is selected from the group consisting of the markers listed in Tables 2-5 and 8-10, and
b) expression of the marker in a second sample obtained from the subject following provision of the portion of the test compound or therapy,
wherein a significantly lower level of expression of the marker in the second sample, relative to the first sample, is an indication that the test compound or therapy is efficacious for inhibiting differentiation of Th1 or Th2 cells in the subject.

28. A method of assessing the efficacy of a test compound or therapy for modulating growth or maturation of Th1 or Th2 cells in a subject, the method comprising comparing:
a) expression of a marker in the first sample obtained from the subject prior to providing at least a portion of the test compound or therapy to the subject, wherein the marker is selected from the group consisting of the markers listed in Tables 2-5 and 8-10 and not including IFNG, SCYA20, or APT1, and
b) expression of the marker in a second sample obtained from the subject following provision of the portion of the test compound or therapy,
wherein a significantly enhanced level of expression of the marker in the second sample, relative to the first sample, is an indication that the test compound or therapy is efficacious for inhibiting growth or maturation of Th1 or Th2 cells in the subject.

29. A method of assessing the efficacy of a test compound or therapy for modulating growth or maturation of Th1 or Th2 cells in a subject, the method comprising comparing:
a) expression of a marker in the first sample obtained from the subject prior to providing at least a portion of the test compound or therapy to the subject, wherein the marker is selected from the group consisting of the markers listed in Tables 2-5 and 8-10 and not including IFNG, SCYA20, or APT1, and
b) expression of the marker in a second sample obtained from the subject following provision of the portion of the test compound or therapy,
wherein a significantly enhanced level of expression of the marker in the first sample, relative to the second sample, is an indication that the test compound or therapy is efficacious for inhibiting growth or maturation of Th1 or Th2 cells in the subject.

30. A method of selecting a composition for modulating differentiation of Th1 or Th2 cells in a subject, the method comprising:
a) obtaining a sample comprising cells from the subject;
b) separately maintaining aliquots of the sample in the presence of a plurality of test compositions;
c) comparing expression of a marker in each of the aliquots, wherein the marker is selected from the group consisting of the markers listed in Tables 2-5 and 8-10; and
d) selecting one of the test compositions which induces a lower level of expression of the marker in the aliquot containing that test composition, relative to other test compositions.

31. A method of selecting a composition for modulating differentiation of Th1 or Th2 cells in a subject, the method comprising:
a) obtaining a sample comprising cells from the subject;
b) separately maintaining aliquots of the sample in the presence of a plurality of test compositions;
c) comparing expression of a marker in each of the aliquots, wherein the marker is selected from the group consisting of the markers listed in Tables 2-5 and 8-10; and
d) selecting one of the test compositions which induces an enhanced level of expression of the marker in the aliquot containing that test composition, relative to other test compositions.

32. A method of selecting a composition for modulating growth or maturation of Th1 or Th2 cells in a subject, the method comprising:
a) obtaining a sample comprising cells from the subject;
b) separately maintaining aliquots of the sample in the presence of a plurality of test compositions;
c) comparing expression of a marker in each of the aliquots, wherein the marker is selected from the group consisting of the markers listed in Tables 2-5 and 8-10 but not including IFNG, SCYA20, or APT1; and
d) selecting one of the test compositions which induces an enhanced level of expression of the marker in the aliquot containing that test composition, relative to other test compositions.

33. A method of selecting a composition for modulating growth or maturation of Th1 or Th2 cells in a subject, the method comprising:
a) obtaining a sample comprising cells from the subject;
b) separately maintaining aliquots of the sample in the presence of a plurality of test compositions;
c) comparing expression of a marker in each of the aliquots, wherein the marker is selected from the group consisting of the markers listed in Tables 2-5 and 8-10 but not including IFNG, SCYA20, or APT1; and
d) selecting one of the test compositions which induces an enhanced level of expression of the marker in the aliquot containing that test composition, relative to other test compositions.

34. A method of modulating differentiation, growth, or development of Th1 or Th2 cells in a subject, the method comprising:
a) obtaining a sample comprising cells from the subject;
b) separately maintaining aliquots of the sample in the presence of a plurality of test compositions;
c) comparing expression of a marker in each of the aliquots, wherein the marker is selected from the group consisting of the markers listed in Tables 2-5 and 8-10; and
d) administering to the subject at least one of the test compositions which induces a lower level of expression of the marker in the aliquot containing that test composition, relative to other test compositions.

35. A method of modulating differentiation, growth, or development of Th1 or Th2 cells in a subject, the method comprising:
a) obtaining a sample comprising cells from the subject;
b) separately maintaining aliquots of the sample in the presence of a plurality of test compositions;
c) comparing expression of a marker in each of the aliquots, wherein the marker is selected from the group consisting of the markers listed in Tables 2-5 and 8-10; and
d) administering to the subject at least one of the test compositions which induces a higher level of expression of the marker in the aliquot containing that test composition, relative to other test compositions.

36. A kit for assessing whether Th1 or Th2 cells are present in a subject, the kit comprising reagents for assessing expression of a marker selected from the group consisting of the markers listed in Tables 2-5 and 8-10.

37. A kit for assessing the presence of mature Th1 or Th2 cells, the kit comprising a nucleic acid probe wherein the probe specifically binds with a transcribed polynucleotide corresponding to a marker selected from the group consisting of the markers listed in Tables 7-11 and not including IFNG, SCYA20, or APT1.

38. A kit for assessing the presence of Th1 or Th2 cells differentiated for 24 or fewer hours, the kit comprising a nucleic acid probe wherein the probe specifically binds with a transcribed polynucleotide corresponding to a marker selected from the group consisting of the markers listed in Tables 1-6 and not including IFNG, SCYA20, or APT1.

39. A kit for assessing the suitability of each of a plurality of compounds for modulating differentiation of Th1 or Th2 cells in a subject, the kit comprising:
a) the plurality of compounds; and
b) a reagent for assessing expression of a marker selected from the group consisting of the markers listed in Tables 2-5 and 8-10.

40. A kit for assessing the presence of Th1 or Th2 cells in a sample, the kit comprising an antibody, wherein the antibody specifically binds with a protein corresponding to a marker selected from the group consisting of the markers listed in Tables 2-5 and 8-10.

41. A kit for assessing the presence of Th1 or Th2 cells in a sample, the kit comprising a nucleic acid probe wherein the probe specifically binds with a transcribed polynucleotide corresponding to a marker selected from the group consisting of the markers listed in Tables 2-5 and 8-10.

42. A method of assessing the potential of a test compound to trigger the differentiation of Th1 or Th2 cells from naïve T cells, the method comprising:
a) maintaining separate aliquots of naïve T cells in the presence and absence of the test compound; and
b) comparing expression of a marker in each of the aliquots, wherein the marker is selected from the group consisting of the markers listed in Tables 2-5 and 8-10,
wherein a significantly enhanced level of expression of the marker in the aliquot maintained in the presence of the test compound, relative to the aliquot maintained in the absence of the test compound, is an indication that the test compound possesses the potential for triggering naïve T cells to differentiate into Th1 or Th2 cells.

43. A method of assessing the potential of a test compound to trigger the differentiation of Th1 or Th2 cells from naïve T cells, the method comprising:
a) maintaining separate aliquots of naïve T cells in the presence and absence of the test compound; and
b) comparing expression of a marker in each of the aliquots, wherein the marker is selected from the group consisting of the markers listed in Tables 2-5 and 8-10,
wherein a significantly decreased level of expression of the marker in the aliquot maintained in the presence of the test compound, relative to the aliquot maintained in the absence of the test compound, is an indication that the test compound possesses the potential for triggering naïve T cells to differentiate into Th1 or Th2 cells.

44. A kit for assessing the potential for triggering the differentiation of naïve T cells into Th1 or Th2 cells, the kit comprising naïve T cells and a reagent for assessing expression of a marker, wherein the marker is selected from the group consisting of the markers listed in Tables 2-5 and 8-10.

45. A method of treating a subject in which differentiation of naïve T cells into Th1 and Th2 cells is desired, the method comprising providing to cells of the subject a protein corresponding to a marker selected from the markers listed in Tables 2-5 and 8-10.

46. The method of claim 45, wherein the protein is provided to the cells by providing a vector comprising a polynucleotide encoding the protein to the cells.

47. A method of treating a subject in which differentiation of naïve T cells into Th1 and Th2 cells is desired, the method comprising providing to cells of the subject an antisense oligonucleotide complementary to a polynucleotide corresponding to a marker selected from the markers listed in Tables 2-5 and 8-10.

48. A method of inhibiting Th1 or Th2 differentiation in a subject, the method comprising inhibiting expression of a gene corresponding to a marker selected from the markers listed in Tables 2-5 and 8-10.

49. A method of inhibiting Th1 or Th2 differentiation in a subject, the method comprising enhancing expression of a gene corresponding to a marker selected from the markers listed in Tables 2-5 and 8-10.
